(19) United States
(12) Patent Application Publication
(10) Pub. No.: US 2008/0262226 A1
(43) Pub. Date: Oct. 23, 2008

(54) METHODS OF MAKING COMPOUNDS HAVING A BETA-ADRENERGIC INHIBITOR AND A LINKER AND METHODS OF MAKING COMPOUNDS HAVING A BETA-ADRENERGIC INHIBITOR, A LINKER AND A PHOSPHODIESTERASE INHIBITOR

(75) Inventor: Gang Chen, Vancouver (CA)

Correspondence Address: SEED INTELLECTUAL PROPERTY LAW GROUP PLLC, 701 FIFTH AVE, SUITE 5400, SEATTLE, WA, 98104, US

(73) Assignee: ARTESIAN THERAPEUTICS, INC., Vancouver (CA)

(21) Appl. No.: 11/936,606

(22) Filed: Nov. 7, 2007

Related U.S. Application Data
(60) Provisional application No. 60/857,662, filed on Nov. 7, 2006.

(51) Int. Cl. C07D 401/02; C07D 211/36; C07D 401/12; C07D 211/56

(52) U.S. Cl. 544/238; 546/226; 546/223

(57) ABSTRACT

A method is provided for making compounds comprising a beta-adrenergic inhibiting moiety and a linking moiety, the method comprising: a) reacting a compound of formula (A): (R¹—(CH—O—CH₂)) with at least one of NH₃, NH₄, NH₄ClNH₃ and R^{12′}NH₂ thereby forming a compound of formula (B): (R¹—CH(OH)—CH₂—NHR^{12′}); and b) reacting a compound of formula (B) with a compound of formula (C): (R³═O), thereby forming a compound of formula (D): (R¹—COH—CH₂—N(R³)(R^{12′})), wherein R¹ comprises the beta-adrenergic inhibiting moiety or comprises the beta-adrenergic inhibiting moiety when bonded to —COH—CH₂—N(R^{12′})— of formula (D); R³ comprises the linking moiety and is bonded to the =0 of formula (C) via a carbon atom; and R^{12′} is selected from hydrogen and a protecting group.

### CROSS-REFERENCE TO RELATED APPLICATION

This application claims the benefit under 35 U.S.C. § 119(e) of U.S. Provisional Patent Application No. 60/857,662, filed Nov. 7, 2006, where this provisional application is incorporated herein by reference in its entirety.

### BACKGROUND

1. Technical Field

This application relates to the field of making chemical compounds and, more specifically, to methods of making compounds having a beta-adrenergic inhibiting moiety and at least a linking moiety.

2. Description of the Related Art

U.S. Pat. No. 5,100,892 describes dihydropyridine compounds having a linking group, processes of synthesis of the dihydropyridine compounds having a linking group and their use to treat hypertension and other cardiac conditions such as congestive heart failure.

Published European Application No. 0412814 describes compounds having cardiotonic and beta blocking activity, methods of making those compounds and their use for treating congestive heart failure.

Published PCT Application Nos. WO 2004/050657, WO 2006/060122, WO 2004/058726 and WO 2006/060127 describe compounds possessing inhibitory activity against beta-adrenergic receptors and inhibitory activity against phosphodiesterase, including type 3 phosphodiesterase. Methods of preparing compounds possessing inhibitory activity against beta-adrenergic receptors and inhibitory activity against phosphodiesterase and methods of using such compounds for treating cardiovascular disease, stroke, epilepsy, ophthalmic disorder or migraine are also described.

U.S. Pat. No. 7,022,732 describes propanolamine derivatives having 1,4-benzodioxane rings and methods of making propanolamine derivatives having 1,4-benzodioxane rings wherein the compounds have a high activity and selectivity to human beta₃-adrenergic receptor as well as have high intrinsic activity. Methods of making those compounds may involve reacting two compounds having the following general formulae:

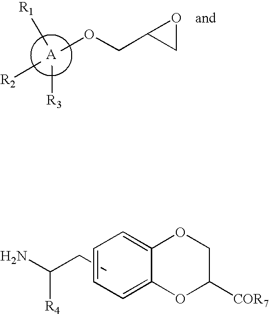

to form a propanolamine derivative having a 1,4-benzodioxane ring. Other methods of making propanolamine derivatives having 1,4-benzodioxane rings are also provided, including reacting two compounds having the following general formulae:

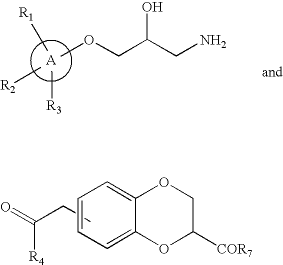

to form a compound having the general formula:

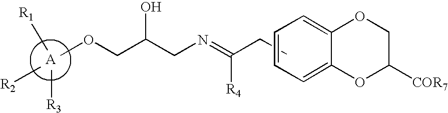

and then reducing such a compound to prepare a propanolamine derivative having a 1,4-benzodioxane ring.

### BRIEF SUMMARY

In various embodiments, there is provided a method of making a compound comprising a beta-adrenergic inhibiting moiety and a linking moiety, the method comprising: a) reacting a compound of formula (A):

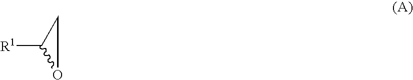

with at least one of NH₃, NH₄OH, ammonium salt (e.g., NH₄X where X=halide), NH₄Cl—NH₃, and a nitrogen-delivering reagent (e.g., NaN₃, KN₃, substituted or unsubstituted benzylamines, arylmethylamines, potassium phthalimide), thereby forming a compound of formula (B) after reduction or deprotection, if required:

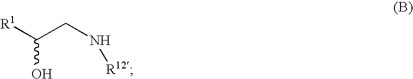

and b) reacting a compound of formula (B) with a compound of formula (C):

R³═O (C),

thereby forming a compound of formula (D):

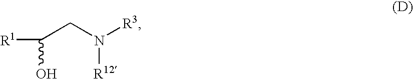

wherein R¹ comprises the beta-adrenergic inhibiting moiety or comprises the beta-adrenergic inhibiting moiety when bonded to

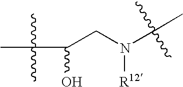

of formula (D); R³ comprises the linking moiety and is bonded to the ═O of formula (C) via a carbon atom; and R^{12′} is selected from hydrogen and a protecting group.

In various embodiments, there is provided a method as described herein wherein a compound of formula (A) is reacted with an excess of at least one of NH₃, NH₄OH, ammonium salts, and NH₄Cl—NH₃; and R^{12′} is H.

In various embodiments, there is provided a method as described herein wherein a compound of formula (A) is reacted with an excess of NH₃ and R12′ is H.

In various embodiments, there is provided a method as described herein wherein formula (A) is racemic

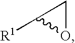

substantially pure

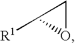

or substantially pure

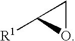

In various embodiments, there is provided a method as described herein wherein R³ is selected from the group consisting of: C₃-C₁₅ aryl, C₃-C₁₅ heteroaryl, C₁-C₁₅ alkyl, C₂-C₁₅ alkenyl, C₂-C₁₅ alkynyl, C₁-C₅ heteroalkyl, C₂-C₅ heteroalkenyl, C₂-C₁₅ heteroalkynyl, C₃-C₁₅ cycloalkyl, C₃-C₁₅ cycloalkenyl, C₃-C₁₅ cycloalkynyl, C₃-C₁₅ heterocycloalkyl, C₃-C₁₅ heterocycloalkenyl, C₃-C₁₅ heterocycloalkynyl, protected C₃-C₁₅ heterocycloalkyl, protected C₃-C₁₅ heterocycloalkenyl, protected C₃-C₁₅ heterocycloalkynyl, C₁-C₁₅ alkoxy and C₃-C₁₅ acylaminoalkyl; and R³ is unsubstituted or substituted with one or more heteroatoms selected from the group consisting of oxygen, sulfur, nitrogen and halogen.

In various embodiments, there is provided a method as described herein wherein R³ is selected from the group consisting of: substituted C₃-C₁₅ heterocycloalkyl, substituted protected C₃-C₁₅ heterocycloalkyl, substituted C₃-C₅ cycloalkyl, substituted protected C₃-C₁₅ cycloalkyl, substituted C₃-C₁₅ heterocycloalkenyl, substituted protected C₃-C₁₅ heterocycloalkenyl, substituted C₃-C₁₅ cycloalkenyl and substituted protected C₃-C₁₅ cycloalkenyl.

In various embodiments, there is provided a method as described herein wherein R³ is a protected C₃-C₁₅ heterocycloalkyl.

In various embodiments, there is provided a method as described herein wherein formula (C) is a compound selected from the group consisting of:

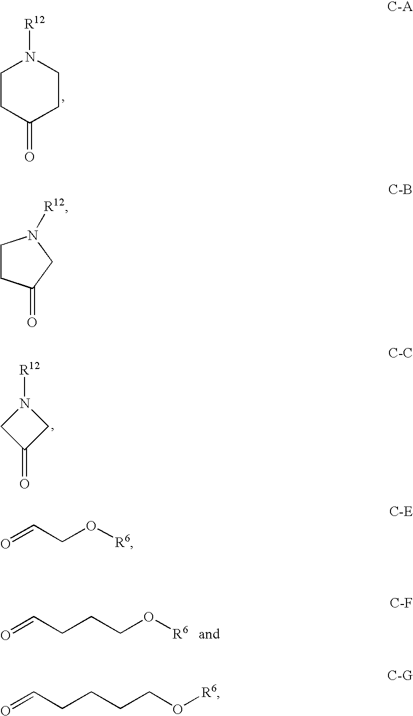

wherein R⁶ comprises a phosphodiesterase inhibiting moiety or comprises a phosphodiesterase inhibiting moiety when bonded to —O— of any one of formulas C-E, C-F and C-G and R¹² is a protecting group.

In various embodiments, there is provided a method as described herein wherein R¹² is selected from the group consisting of: Boc, Fmoc, Bn and Cbz.

In various embodiments, there is provided a method as described herein wherein R⁶ is selected from the group consisting of:

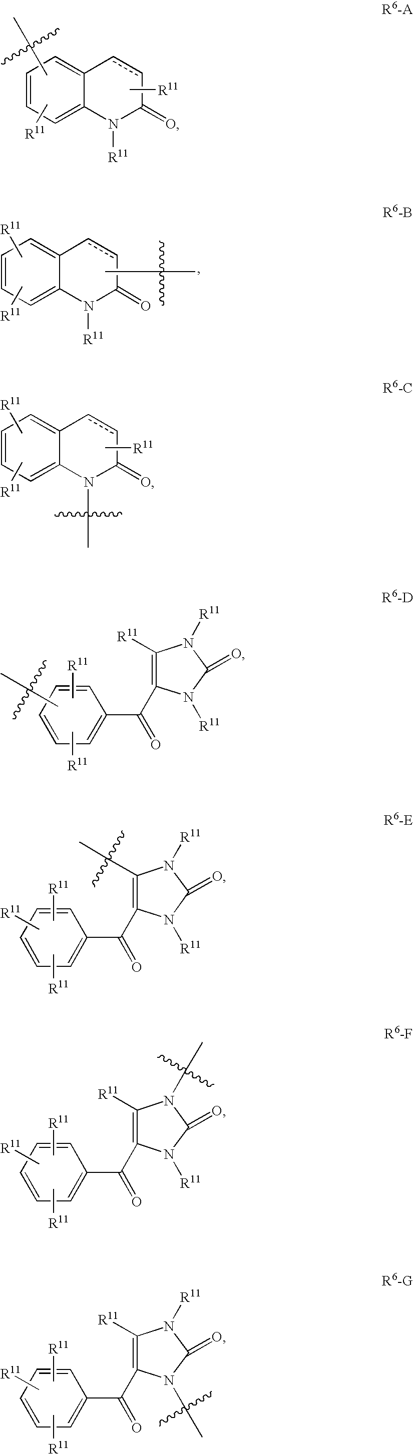

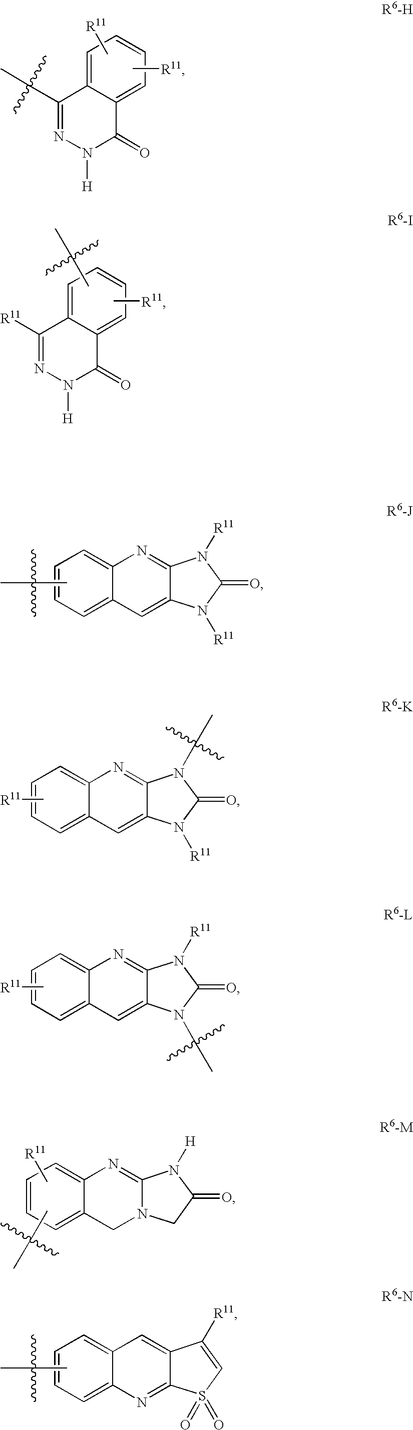

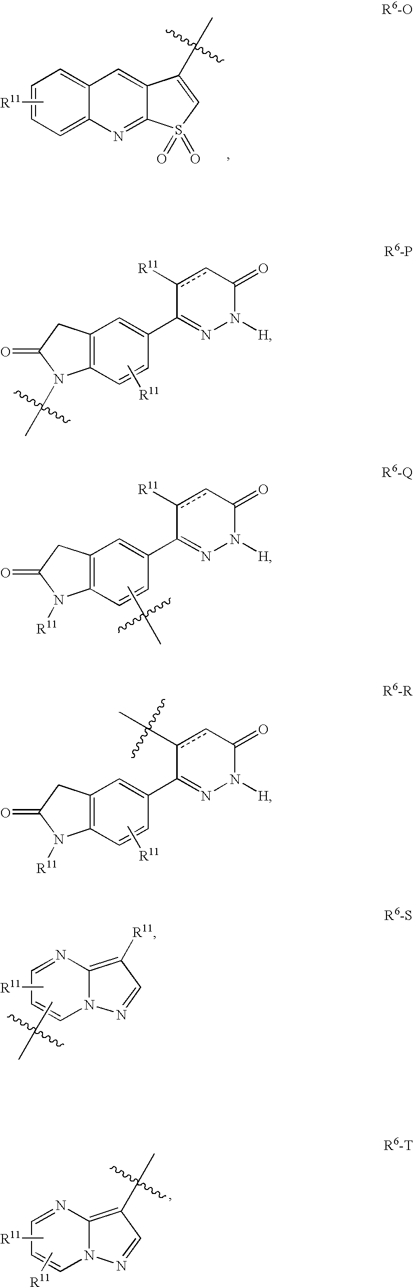

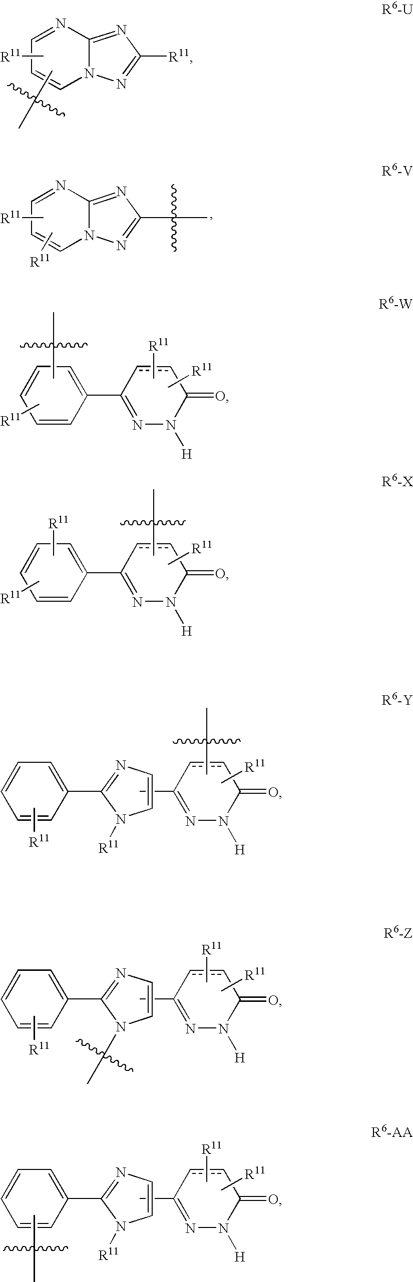

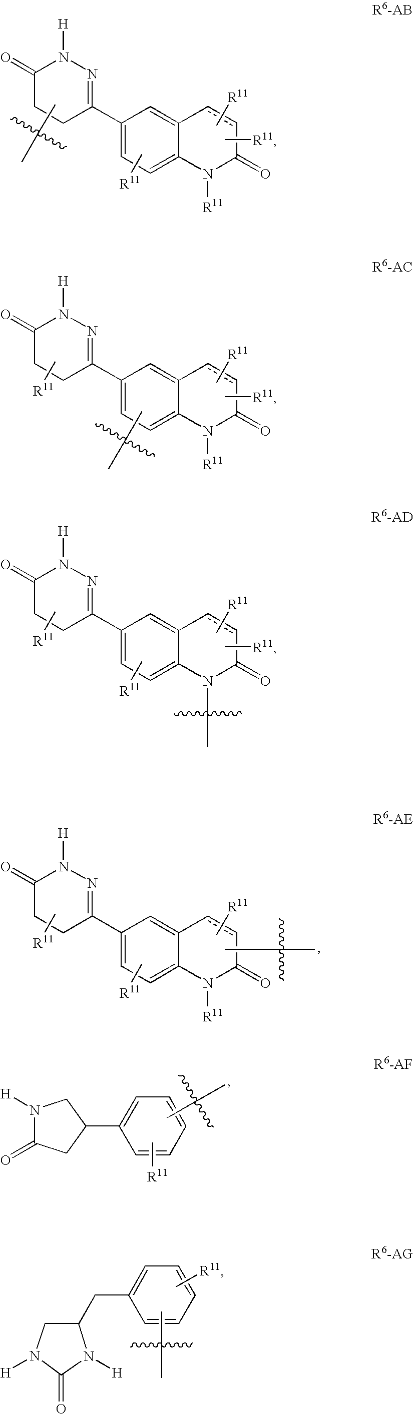

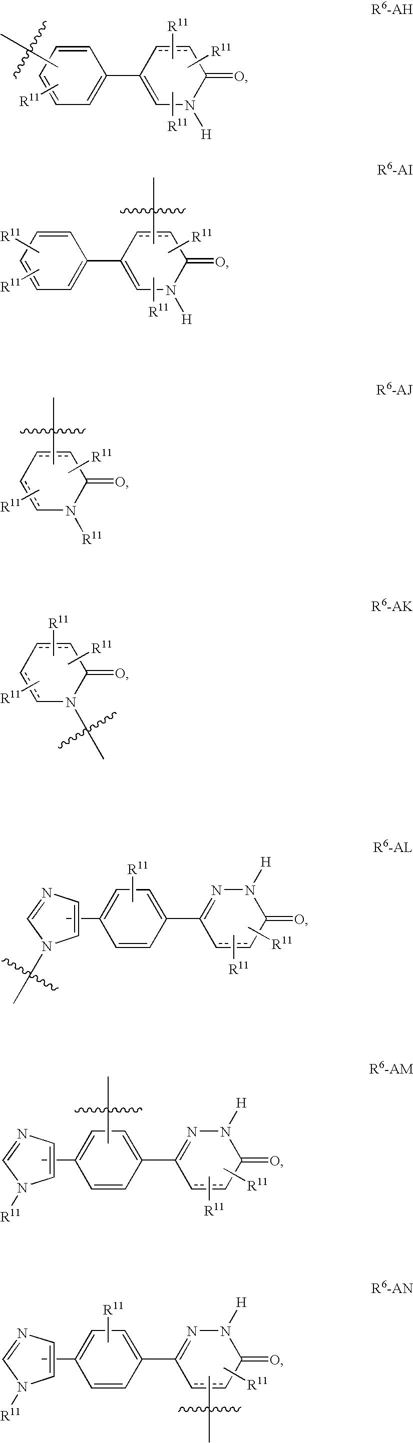

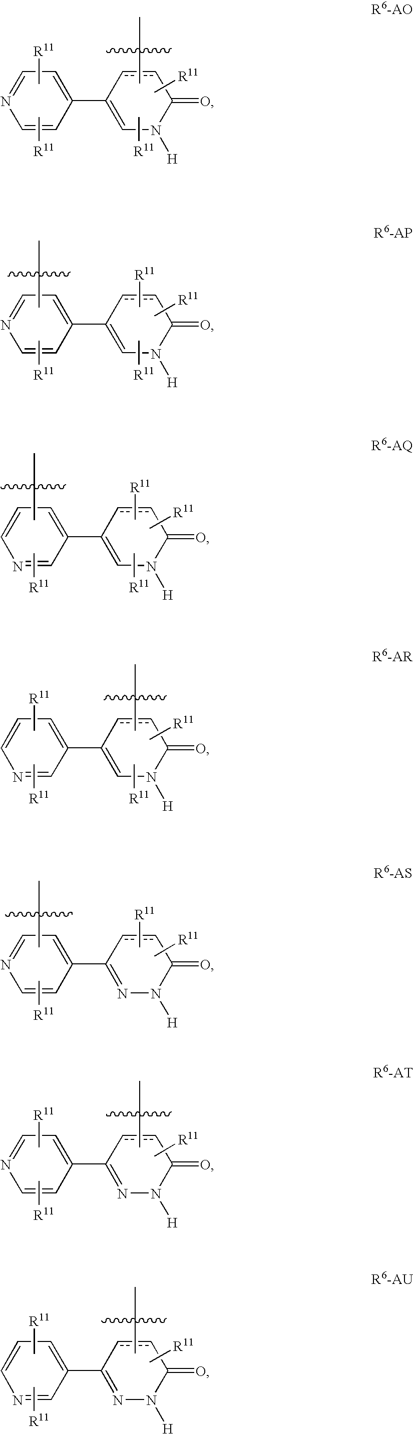

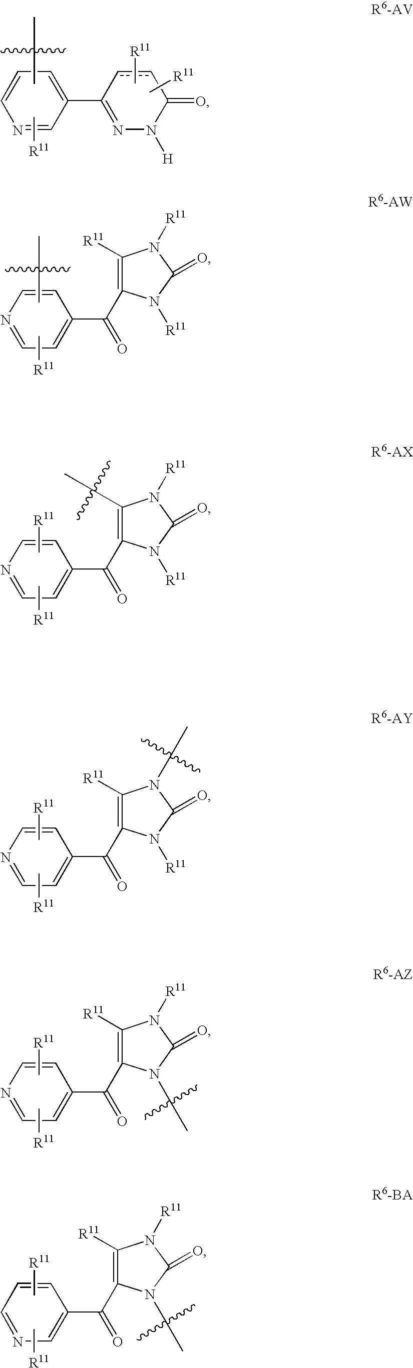

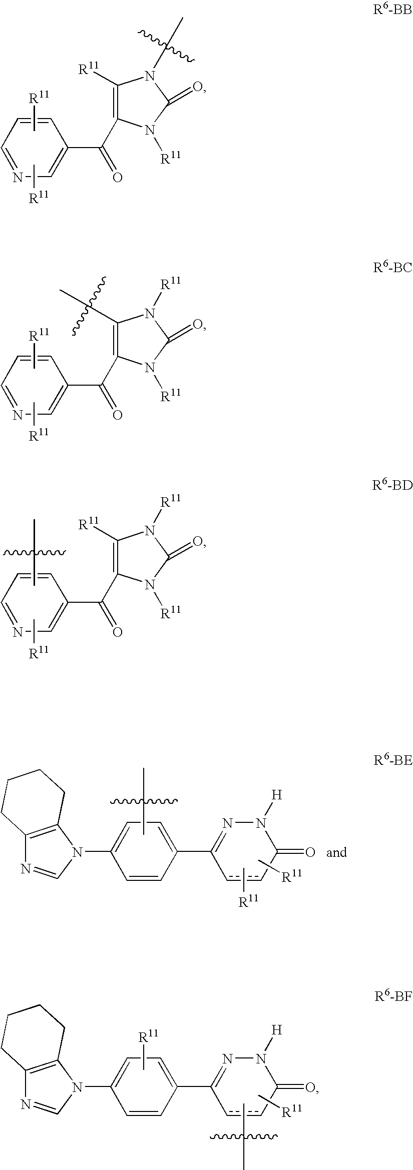

wherein each R¹¹ is independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₁-C₁₅ alkoxy and C₃-C₁₅ acylaminoalkyl.

In various embodiments, there is provided a method as described herein wherein R⁶ is:

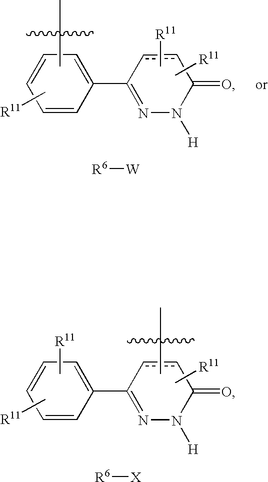

wherein each R¹¹ is independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₁-C₁₅ alkoxy and C₃-C₁₅ acylaminoalkyl.

In various embodiments, there is provided a method as described herein wherein each R¹¹ is independently selected from the group consisting of: a hydrogen radical and a halo.

In various embodiments, there is provided a method as described herein wherein R⁶ is:

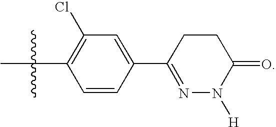

In various embodiments, there is provided a method as described herein wherein R³ is selected from the group consisting of:

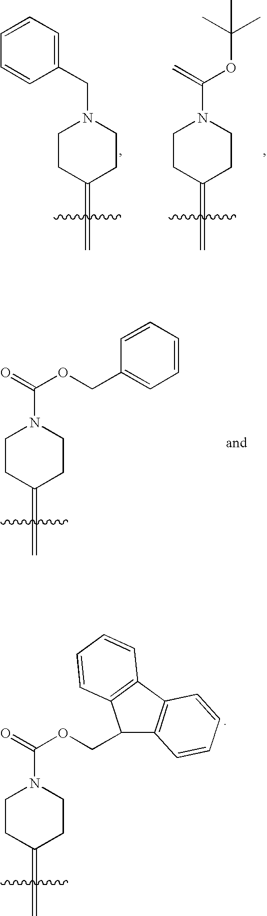

In various embodiments, there is provided a method as described herein wherein R³ is:

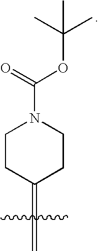

In various embodiments, there is provided a method as described herein wherein R¹ is a moiety of formula (E):

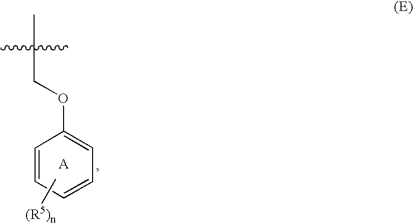

wherein n is an integer selected from 1, 2, 3, 4 and 5; each R⁵ is independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₃-C₁₅ heterocycloalkyl, C₃-C₁₅ heterocycloalkenyl, C₃-C₁₅ heterocycloalkynyl, protected C₃-C₁₅ heterocycloalkyl, protected C₃-C₁₅ heterocycloalkenyl, protected C₃-C₁₅ heterocycloalkynyl, C₁-C₁₅ alkoxy and C₃-C₁₅ acylaminoalkyl; each R⁵ is independently unsubstituted or substituted with one or more heteroatoms selected from the group consisting of oxygen, sulfur, nitrogen and halogen; and each R⁵ has one or more points of attachment to ring A.

In various embodiments, there is provided a method as described herein wherein R¹ is selected from the group consisting of:

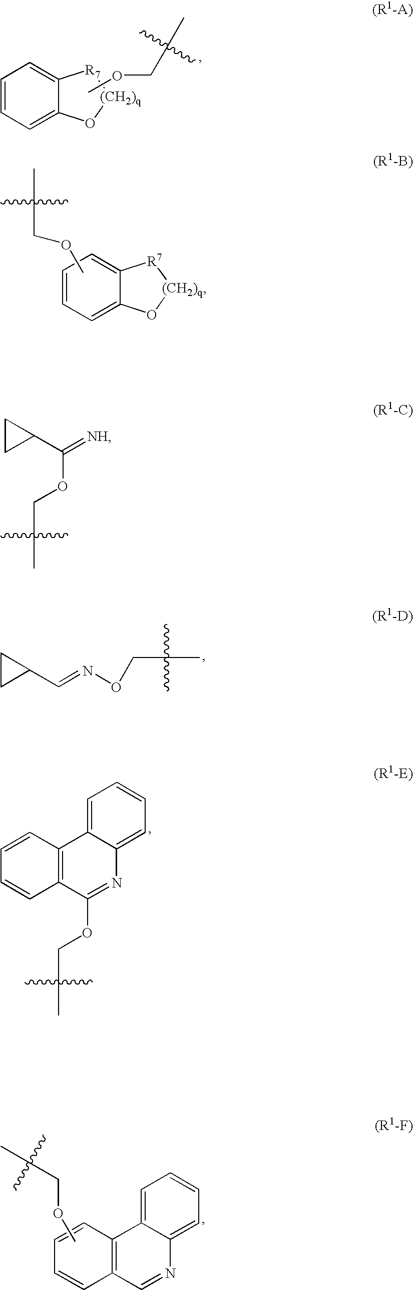

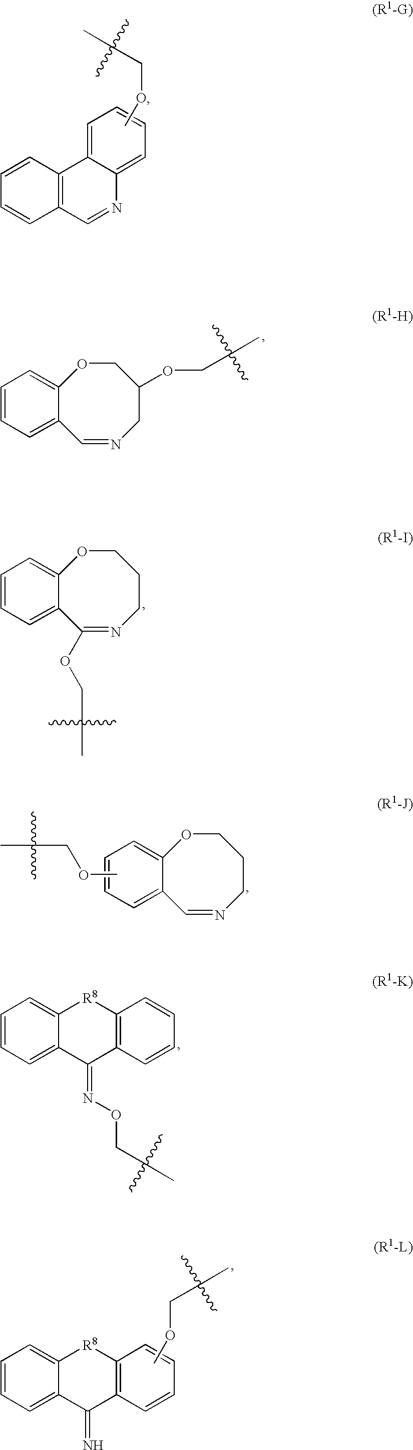

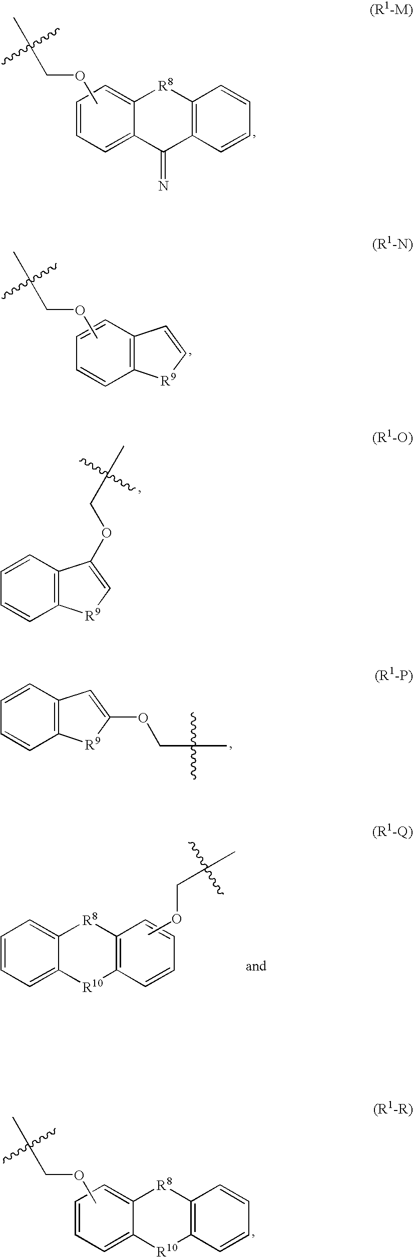

wherein q is an integer selected from 1, 2 and 3, R⁷ is O, C═O, S, NH or CH₂, R⁸ is a bond, —CH₂—, —CH═, —CH₂CH₂—, —CH═CH—, O, S or NH, R⁹ is —CH₂—, —CH(R⁵)—, —C(R⁵)(R⁵)—, O, S NH or N(R⁵), and R¹⁰ is a bond or O, where each R⁵ is as defined above.

In various embodiments, there is provided a method as described herein wherein n is 1 and R⁵ of formula (E) is selected from the group consisting of: a C₆-C₇ heterocycloalkyl having two points of attachment to ring A, halo and cyano.

In various embodiments, there is provided a method as described herein wherein R¹ is selected from the group consisting of:

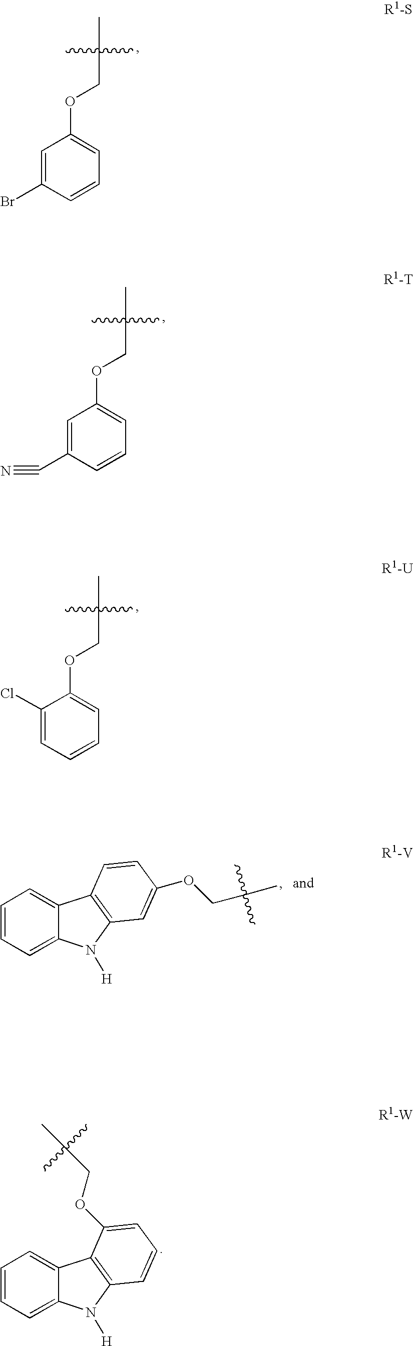

In various embodiments, there is provided a method of making a compound comprising a beta-adrenergic inhibiting moiety and a linking moiety, the method comprising: a) reacting a compound of formula (A):

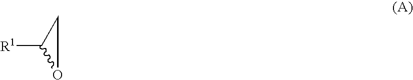

with at least one of NH₃, NH₄OH, ammonium salts, and NH₄Cl—NH₃, thereby forming a compound of formula (B):

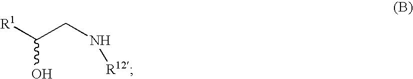

b) reacting a compound of formula (B) with a compound of formula (C) under reductive conditions (such as in the presence of NaB(OAc)₃H, NaCNBH₃, LiCNBH₃, NaBH₄/Lewis acid, or Me₄N(OAc)₃BH):

R³═O (C),

thereby forming a compound of formula (D):

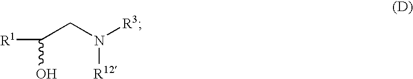

and c) reacting a compound of formula (D) with a compound of formula (F) wherein LG is a leaving group such as hydroxyl, or where formula (F) is in an activated form (such as an acyl halide or acyl anhydride):

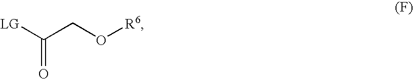

thereby forming a compound of formula (G):

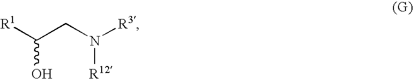

wherein R¹ comprises the beta-adrenergic inhibiting moiety or comprises the beta-adrenergic inhibiting moiety when bonded to

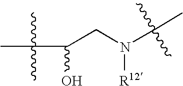

of formula (D); R³ comprises the linking moiety and is bonded to the ═O in formula (C) via a carbon atom; R³ is substituted R³, often substituted with

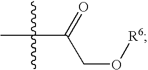

and R⁶ comprises a phosphodiesterase inhibiting moiety or comprises a phosphodiesterase inhibiting moiety when bonded to —O— in R^{3′}.

In various embodiments, there is provided a method as described herein wherein R³ is selected from the group consisting of: C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₃-C₁₅ heterocycloalkyl, C₃-C₁₅ heterocycloalkenyl, C₃-C₁₅ heterocycloalkynyl, C₁-C₁₅ alkoxy and C₃-C₁₅ acylaminoalkyl; and R³ is unsubstituted or substituted with one or more heteroatoms selected from the group consisting of oxygen, sulfur, nitrogen and halogen.

In various embodiments, there is provided a method as described herein further comprising removing a protecting group from a compound of formula (D), thereby forming a deprotected compound of formula (D), prior to reacting the deprotected compound of formula (D) with the compound of formula (F).

In various embodiments, there is provided a method as described herein further comprising removing a protecting group from a compound of formula (G), thereby forming a deprotected compound of formula (G).

In various embodiments, there is provided a method as described herein further comprising removing a protecting group from a compound of formula (D), thereby forming a deprotected compound of formula (D), prior to reacting the deprotected compound of formula (D) with the compound of formula (F) and wherein R³ is selected from the group consisting of: protected C₃-C₁₅ heteroaryl, protected C₁-C₁₅ heteroalkyl, protected C₂-C₁₅ heteroalkenyl, protected C₂-C₁₅ heteroalkynyl, protected C₃-C₁₅ heterocycloalkyl, protected C₃-C₁₅ heterocycloalkenyl, protected C₃-C₁₅ heterocycloalkynyl and protected C₃-C₁₅ acylaminoalkyl; and R³ is unsubstituted or substituted with one or more heteroatoms selected from the group consisting of oxygen, sulfur, nitrogen and halogen.

In various embodiments, there is provided a method as described herein wherein R³ is selected from the group consisting of: protected C₃-C₁₅ heterocycloalkyl, protected C₃-C₁₅ cycloalkyl, protected C₃-C₁₅ heterocycloalkenyl and protected C₃-C₁₅ cycloalkenyl; and R³ is unsubstituted or substituted with one or more heteroatoms selected from the group consisting of oxygen, sulfur, nitrogen and halogen.

In various embodiments, there is provided a method as described herein wherein R³ is a protected C₃-C₁₅ heterocycloalkyl.

In various embodiments, there is provided a method as described herein wherein R³ is selected from the group consisting of:

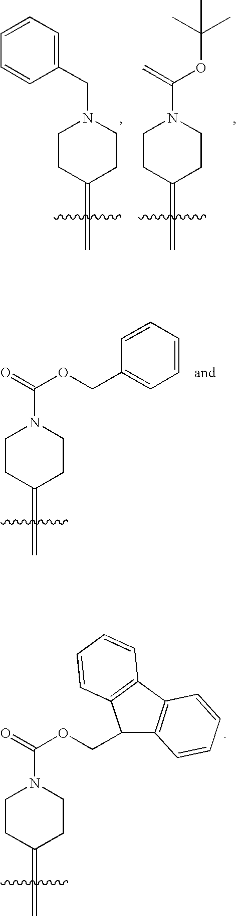

In various embodiments, there is provided a method as described herein wherein formula (C) is a compound selected from the group consisting of:

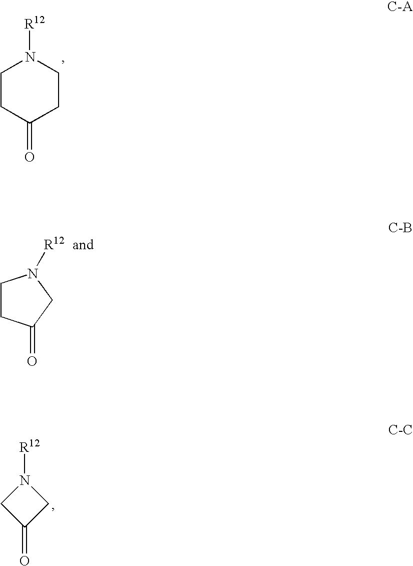

wherein R¹² is a protecting group.

In various embodiments, there is provided a method as described herein wherein the protecting group is a tert-butoxycarbonyl group.

In various embodiments, there is provided a method as described herein wherein R³ is:

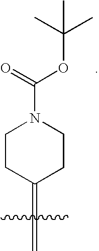

In various embodiments, there is provided a method of making a compound comprising a beta-adrenergic inhibiting moiety and a linking moiety, the method comprising: a) reacting

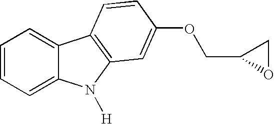

with an excess of NH₃, thereby forming

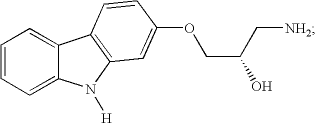

and b) reacting under reductive conditions

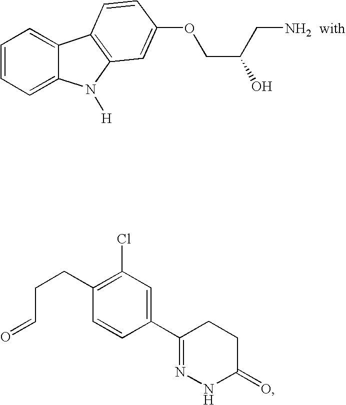

thereby forming

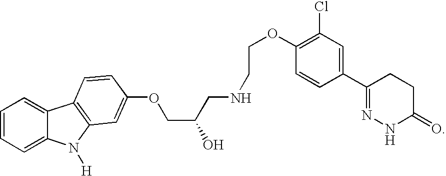

In various embodiments, there is provided a method of making a compound comprising a beta-adrenergic inhibiting moiety and a linking moiety, the method comprising: a) reacting

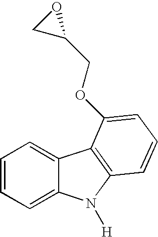

with an excess of NH₃, thereby forming

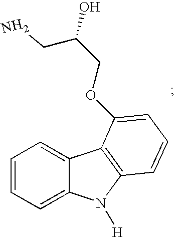

and b) reacting under reductive conditions

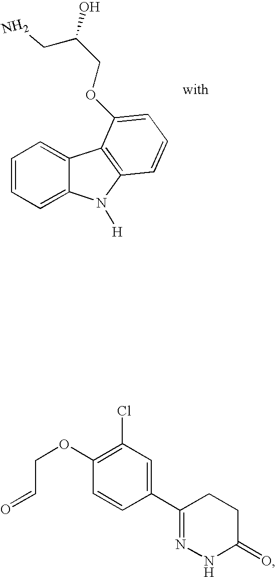

thereby forming

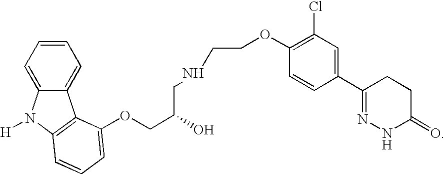

In various embodiments, there is provided a method of making a compound comprising a beta-adrenergic inhibiting moiety and a linking moiety, the method comprising: a) reacting

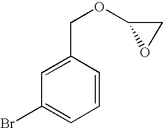

with an excess of NH₃, thereby forming

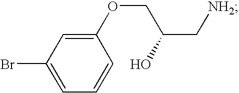

b) reacting under reductive conditions

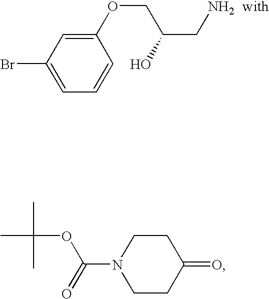

thereby forming

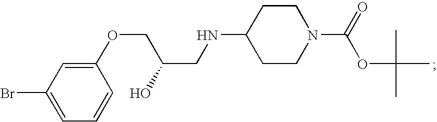

c) reacting

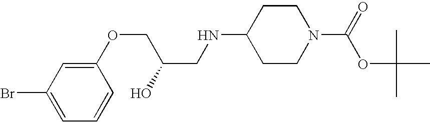

with a strong organic or inorganic acid (such as HCl) is a suitable solvent (such as THF) thereby forming

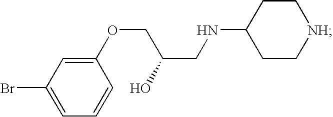

and d) reacting

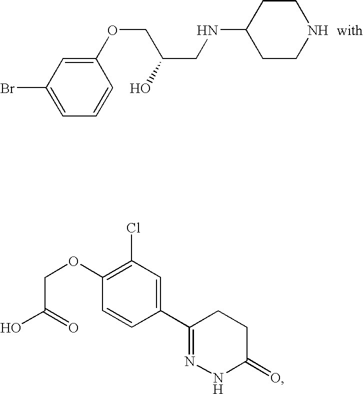

thereby forming

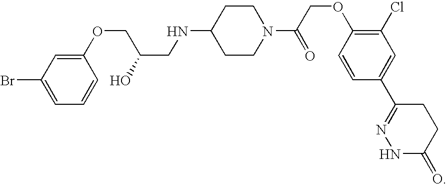

In various embodiments, there is provided a method of making a compound comprising a beta-adrenergic inhibiting moiety and a linking moiety, the method comprising: a) reacting

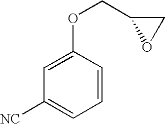

with an excess of NH₃, thereby forming

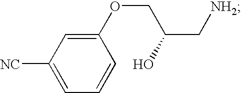

b) reacting under reductive conditions

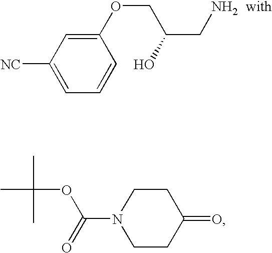

thereby forming

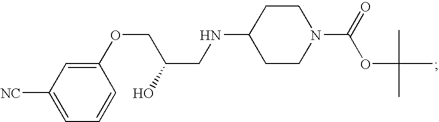

c) reacting

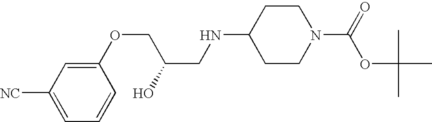

with, for example, HCl-THF, thereby forming

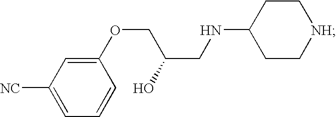

and d) reacting

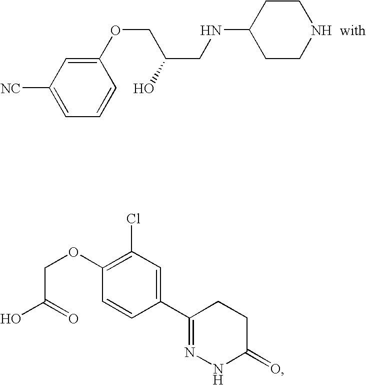

thereby forming

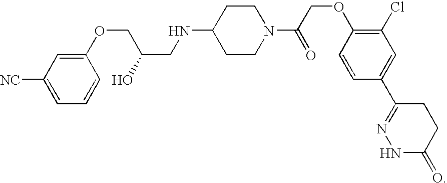

In various embodiments, there is provided a method of making a compound comprising a beta-adrenergic inhibiting moiety and a linking moiety, the method comprising: a) reacting

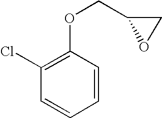

with an excess of NH₃, NH₄OH, ammonium salts, or NH₄Cl—NH₃, thereby forming

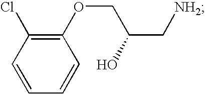

b) reacting under reductive conditions

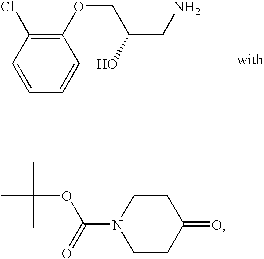

thereby forming

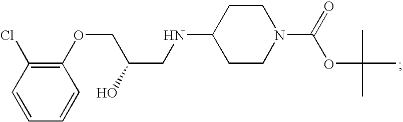

c) reacting

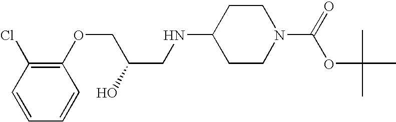

with, for example, HCl-THF thereby forming

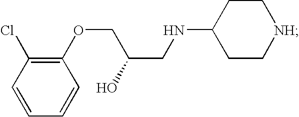

and d) reacting

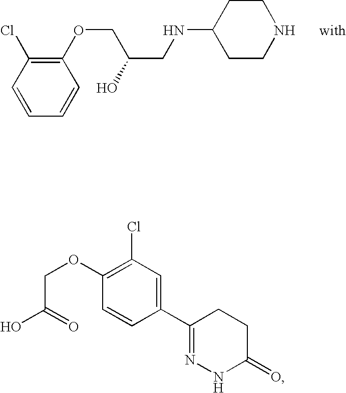

thereby forming

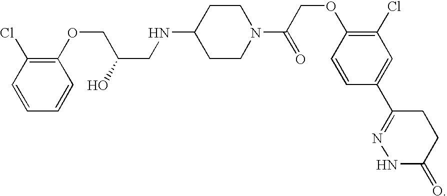

### DETAILED DESCRIPTION

Various embodiments described herein may or may not be based, in part, on the discovery that making compounds described herein by opening an epoxide with ammonia, or an ammonium salt, followed by a reductive amination step provides a secondary amine, which secondary amine may provide compounds that may or may not be: easy to crystallize, compounds having substantially optically pure chiral centers and/or permit the use of cheaper and/or more widely available reagents to make the secondary amine. Secondary amines made using methods described herein may or may not reduce by-product formation and may or may not reduce the number of purification steps (e.g., column chromatography) required to make purified compounds made by methods described herein.

Methods described herein may be used to make: compounds used in the production of pharmaceutical compositions, pharmaceutically active compounds or both. Some uses of the compounds made by methods described herein are described in European patent application published as 0412814 and international patent applications published as: WO 2004/050657, WO 2006/060122, WO 2004/058726 and WO 2006/060127.

As used herein, a bond shown as “------” refers to a bond that may be present or absent. In some instances, such a bond may be shown in combination with a solid bond [symbol] which indicates that at least the solid bond (i.e., a single bond in the subject bond) is present and the dashed bond (i.e., a double bond in the subject bond) may be present or absent.

As used herein, a bond shown as [symbol] refers to a bond that represents any possible stereo configuration. Such a bond represents the possibility of either an (R)-configuration or an (S)-configuration, but may represent an (R)-configuration or an (S)-configuration. In some instances, the subject bond may represent a racemic mixture of the possible stereo configurations or non-racemic mixtures of the possible stereo configurations.

As used herein, a bond shown as [symbol] refers to a bond that is a point of attachment to a second moiety. Generally, such a bond will have a structure described on one side of the bond and the structure is attached to the second moiety via the subject bond, which second moiety may not be shown in whole or in part as being attached to the subject bond.

As used herein, the term “point of attachment” refers to a bond between two atoms by which an atom of a first chemical entity is attached to an atom of a second chemical entity. Chemical entities may have a single point of attachment to another single chemical entity, multiple points of attachment to another single chemical entity, single points of attachment to multiple other chemical entities, multiple points of attachments to multiple other chemical entities or a combination thereof. An example comprises or includes, but is not limited to, a double bond between two carbons is a single point of attachment. Another example comprises or includes, but is not limited to, two single bonds between a single carbon atom and two different carbon atoms are two points of attachment. A further example comprises or includes, but is not limited to, a first alkane having a bond from a first carbon atom to a second carbon atom on a second alkane comprises a first point of attachment and bond from a third carbon on the first alkane to a fourth carbon on the second alkane comprises a second point of attachment.

As used herein, the term “beta-adrenergic inhibiting moiety” refers to a moiety that inhibits the interaction of catecholamines, norepinephrine, epinephrine and sympathomimetic drugs, like isoproterenol, with beta-adrenergic receptors. All types and subtypes of beta-adrenergic inhibiting moieties are encompassed by this term. Any moiety that completely or partially inhibits of any one or more of beta₁-, beta₂- and beta₃-adrenergic receptors is encompassed by the term “beta-adrenergic inhibiting moiety”. Standard radiolabel-probe-assays to ascertain the beta-adrenergic inhibiting activity of a particular compound are known to a person of skill in the art and may be contracted from commercial organizations, such as CEREP. Alternatively, beta-adrenergic inhibiting moieties may be identified by a person of skill in the art by conducting routine experiments to show that the beta-adrenergic inhibiting moiety reduces or prevents positive chronotropic and positive inotropic effects on the heart when isobuteranol is administered to the subject. Beta-adrenergic inhibiting moieties may have other effects in addition to the blockade of normal isobuteranol-mediated sympathetic actions. Examples of beta-adrenergic inhibiting moieties comprise or include, but are not limited to, metoprolol, carvedilol, 5-demethylbupranolol, propranolol, naolol, timolol, oxprenolol, pindolol, alprenolol, atenolol and acebutolol. Beta-adrenergic inhibiting moieties are also described in more detail below.

As used herein, the term “linking moiety” refers to a moiety that is capable of forming at least one covalent bond or has at least one covalent bond with each of at least two separate moieties. A linking moiety may have a covalent bond with a first moiety and be capable of forming a covalent bond with a second linking moiety or may have a covalent bond with the first moiety and a covalent bond with the second moiety. Generally, a linking moiety does not provide a therapeutic effect when not covalently bonded to another moiety. Linking moieties are also described in more detail below.

As used herein, the term “phosphodiesterase inhibiting moiety” refers to a moiety that inhibits an enzyme capable of hydrolyzing a phosphodiester bond. All types and subtypes of phosphodiesterase inhibiting moieties are contemplated, that is any moiety that completely or partially inhibits of any one or more of phosphodiesterase-1, phosphodiesterase-2, phosphodiesterase-3, phosphodiesterase-4 and phosphodiesterase-5 and other phosphodiesterase types and subtypes is encompassed by the term “phosphodiesterase inhibiting moiety”. A non-limiting example of such an enzyme is an enzyme that is capable of hydrolyzing cAMP to AMP. Standard competitive inhibition assays to determine the phosphodiesterase inhibition activity of a particular compound are known to a person of skill in the art and may be contracted from commercial organizations, such as CEREP. Alternatively, a person of skill in the art is able to identify a phosphodiesterase inhibiting moiety by conducting routine experiments to show that the phosphodiesterase inhibiting moiety induces positive inotropic effects on the heart of a subject when administered to the subject. Non-limiting examples of phosphodiesterase inhibiting moieties comprise or include caffeine, theophylline, amrinone, milrinone, enoximone, piroximone, saterinone, pimobendan, adibendan, sulmazole, levosimendan and rolipram. Phosphodiesterase inhibiting moieties are also described in more detail below.

As used herein the term “heteroatom” refers to any atom that is not carbon or hydrogen. Examples of heteroatoms comprise or include, but are not limited to, nitrogen, sulfur, oxygen, phosphorus, boron, chlorine, fluorine, bromine and iodine.

As used herein, the term “halogen” refers to a fluorine, chlorine, bromine or iodine atom. As used herein, the term “halo” refers to a fluoro, chloro, bromo or iodo radical.

As used herein, the term “alkyl” refers to a saturated straight or branched chain hydrocarbon radical. Examples comprise or include without limitation methyl, ethyl, propyl, iso-propyl, butyl, iso-butyl, tert-butyl, n-pentyl and n-hexyl. As used herein, the term “C₁-C₁₅ alkyl” refers to an alkyl having 1, 2, 3, 4, 5, 6, 7, 8, 9, 10, 11, 12, 13, 14 or 15 carbon atoms.

As used herein, the term “heteroalkyl” refers to an alkyl having one or more heteroatoms in place of a carbon or hydrogen of the alkyl. As used herein, the term “C₁-C₁₅ heteroalkyl” refers to a heteroalkyl having 1, 2, 3, 4, 5, 6, 7, 8, 9, 10, 11, 12, 13, 14 or 15 carbon atoms and heteroatoms. As a non-limiting example, a C₉ heteroalkyl may have 7 carbon atoms, 1 chlorine atom and 1 nitrogen atom.

As used herein, the term “alkenyl” refers to an unsaturated straight or branched chain hydrocarbon radical comprising at least one carbon to carbon double bond. Examples comprise or include without limitation ethenyl, propenyl, iso-propenyl, butenyl, iso-butenyl, tert-butenyl, n-pentenyl and n-hexenyl. As used herein, the term “C₂-C₁₅ alkenyl” refers to an alkenyl having 2, 3, 4, 5, 6, 7, 8, 9, 10, 11, 12, 13, 14 or 15 carbon atoms.

As used herein, the term “heteroalkenyl” refers to an alkenyl having one or more heteroatoms in place of a carbon or hydrogen of the alkenyl. As used herein, the term “C₂-C₁₅ heteroalkenyl” refers to a heteroalkenyl having 2, 3, 4, 5, 6, 7, 8, 9, 10, 11, 12, 13, 14 or 15 carbon atoms and heteroatoms. As a non-limiting example, a C₈ heteroalkenyl may have 5 carbon atoms and 3 chlorine atoms.

As used herein, the term “alkynyl” refers to an unsaturated straight or branched chain hydrocarbon radical comprising at least one carbon to carbon triple bond. Examples comprise or include without limitation ethynyl, propynyl, butynyl, iso-butynyl, pentynyl and hexynyl. As used herein, the term “C₂-C₁₅ alkynyl” refers to an alkynyl having 2, 3, 4, 5, 6, 7, 8, 9, 10, 11, 12, 13, 14 or 15 carbon atoms.

As used herein, the term “heteroalkynyl” refers to an alkynyl having one or more heteroatoms in place of a carbon or hydrogen of the alkynyl. As used herein, the term “C₂-C₁₅ heteroalkynyl” refers to a heteroalkynyl having 2, 3, 4, 5, 6, 7, 8, 9, 10, 11, 12, 13, 14 or 15 carbon atoms and heteroatoms. As a non-limiting example, a C₇ heteroalkynyl may have 5 carbon atoms and 2 chlorine atoms.

As used herein, the term “alkoxy” refers to an alkyl bonded through an oxygen linkage. As used herein, the term “C₁-C₁₅ alkoxy” refers to an alkoxy having 1, 2, 3, 4, 5, 6, 7, 8, 9, 10, 11, 12, 13, 14 or 15 carbon atoms and I oxygen radical. As a non-limiting example, a C₇ alkoxy may have 7 carbon atoms and an oxygen radical.

As used herein, the term “acylaminoalkyl” refers to a heteroalkyl that comprises at least one amino group (i.e., R₃N, R₂NH or RNH₂) and at least one acyl (i.e., RCO) group, where R, R₂ or R₃ is the remainder of the acylaminoalkyl. As used herein, the term “C₃-C₁₅ acylaminoalkyl” refers to an acylaminoalkyl having 3, 4, 5, 6, 7, 8, 9, 10, 11, 12, 13, 14 or 15 carbon atoms and heteroatoms. For example a C₆ acylaminoalkyl may have 4 carbon atoms, 1 nitrogen atom and 1 oxygen atom.

As used herein, the term “cycloalkyl” refers to a mono- or poly-cyclic alkyl radical. Examples comprise or include without limitation, cyclobutyl, cyclopentyl, cyclohexyl, cycloheptyl and cyclooctyl. As used herein, the term “C₃-C₁₅ cycloalkyl” refers to a cycloalkyl having 3, 4, 5, 6, 7, 8, 9, 10, 11, 12, 13, 14 or 15 carbon atoms.

As used herein, the term “cycloalkenyl” refers to a mono- or poly-cyclic alkenyl radical comprising at least one carbon to carbon double bond. Examples comprise or include without limitation, cyclobutenyl, cyclopentenyl, cyclohexenyl, cycloheptenyl and cyclooctenyl. As used herein, the term “C₃-C₁₅ cycloalkenyl” refers to a cycloalkenyl having 3, 4, 5, 6, 7, 8, 9, 10, 11, 12, 13, 14 or 15 carbon atoms.

As used herein, the term “cycloalkynyl” refers to a mono- or poly-cyclic alkynyl radical comprising at least one carbon to carbon triple bond. Examples comprise or include without limitation, cyclobutynyl, cyclopentynyl, cyclohexynyl, cycloheptynyl and cyclooctynyl. As used herein, the term “C₃-C₁₅ cycloalkynyl” refers to a cycloalkynyl having 3, 4, 5, 6, 7, 8, 9, 10, 11, 12, 13, 14 or 15 carbon atoms.

As used herein, the term “heterocycloalkyl” refers to a cycloalkyl having one or more heteroatoms in place of a carbon or hydrogen of the cycloalkyl. As used herein, the term “C₃-C₁₅ heterocycloalkyl” refers to a heterocycloalkyl having 3, 4, 5, 6, 7, 8, 9, 10, 11, 12, 13, 14 or 15 carbon atoms and heteroatoms. As a non-limiting example, a C₇ heterocycloalkyl may have 5 carbon atoms and 1 chlorine atom and one nitrogen atom.

As used herein, the term “heterocycloalkenyl” refers to a cycloalkenyl having one or more heteroatoms in place of a carbon or hydrogen of the cycloalkenyl. As used herein, the term “C₃-C₁₅ heterocycloalkenyl” refers to a heterocycloalkenyl having 3, 4, 5, 6, 7, 8, 9, 10, 11, 12, 13, 14 or 15 carbon atoms and heteroatoms. As a non-limiting example, a C₆ heterocycloalkenyl may have 4 carbon atoms and 2 chlorine atoms.

As used herein, the term “heterocycloalkynyl” refers to a cycloalkynyl having one or more heteroatoms in place of a carbon or hydrogen of the cycloalkynyl. As used herein, the term “C₃-C₁₅ heterocycloalkynyl” refers to a heterocycloalkynyl having 3, 4, 5, 6, 7, 8, 9, 10, 11, 12, 13, 14 or 15 carbon atoms and heteroatoms. As a non-limiting example, a C₆ heterocycloalkynyl may have 4 carbon atoms, 1 chlorine atom and 1 nitrogen atom.

As used herein, the term “aryl” refers to a cyclic aromatic hydrocarbon moiety having one or more closed ring(s). Examples comprise or include without limitation phenyl, benzyl, naphthyl anthracenyl, phenanthracenyl and biphenyl. As used herein, the term “C₃-C₁₅ aryl” refers to an aryl having 3, 4, 5, 6, 7, 8, 9, 10, 11, 12, 13, 14 or 15 carbon atoms.

As used herein, the term “heteroaryl” refers to a cyclic aromatic moiety having one or more closed rings with one or more heteroatom(s) in at least one ring. Examples comprise or include without limitation pyrryl, furanyl, thienyl, pyridinyl, oxazolyl, thiazolyl, benzofuranyl, benzothienyl and benzofuranyl. As used herein, the term “C₃-C₁₅ heteroaryl” refers to a heteroaryl having 3, 4, 5, 6, 7, 8, 9, 10, 11, 12, 13, 14 or 15 carbon atoms and heteroatoms. As a non-limiting example, a C₆ heteroaryl may have 5 carbon atoms and 1 nitrogen atom.

As used herein, the term “substituted” refers to one or more substituents (which may be the same or different), each replacing a chemical entity, which chemical entity is often, but not limited to, a hydrogen atom. Examples of substituents comprise or include without limitation alkyl, heteroalkyl, alkenyl, heteroalkenyl, alkynyl, heteroalkynyl, alkyloxy, acylaminoalkyl, cycloalkyl, heterocylcoalkyl, cycloalkenyl, heterocycloalkenyl, cycloalkynyl, heterocycloalkynyl, aryl, heteroaryl, cyano, nitro, mercapto, halo, hydroxyl, amino, carbonyl, carbamido, carbamyl, carboxyl, thioureido, thiocyanato, sulfoamido, wherein alkyl, alkenyl, alkyloxy, aryl, heteroaryl cycloalkyl and heterocycloalkyl are optionally substituted with alkyl, aryl, heteroaryl, halo, hydroxyl, amino, mercapto, cyano or nitro.

As used herein, the term “protected” refers to a chemical entity that is rendered less reactive or non-reactive by the presence of a protecting group. A protecting group, as used herein, refers to a group that selectively prevents reaction of the protected chemical entity, by temporarily masking or changing the chemistry of a chemical entity that is protected by the protecting group, while allowing other chemical entities in the same molecule to be reacted without affecting the protected chemical entity. Examples of protecting groups comprise or include, but are not limited to, Boc, Fmoc, Cbz and Bn. Various methods of protecting chemical entities and methods of removing protecting groups as well as the composition of many protecting groups are known to a person of skill in the art and are described in the second edition of “Protective Groups in Organic Synthesis”, third edition, by Greene and Wuts (John Wiley & Sons, Inc, 1999).

As used herein, the term “deprotected compound of formula” refers to a compound that has a structure encompassed by the formula and was obtained by removing a protecting group from a first compound, which first compound may or may not have a structure encompassed by the formula.

The following are abbreviations that may be used herein: Boc=tert-butoxycarbonyl; Fmoc=9-Fluorenylmethoxycarbonyl; Bn=Benzyl; Cbz=benzyloxycarbonyl; THF=tetrahydrofurane; cAMP=cyclic adenosine monophosphate; AMP=adenosine monophosphate; OAc=acetate or acetyl; HOAc=acetic acid; EDCI=1-(3-dimethylaminopropyl)-3-ethylcarbodimide hydrochloride; HOBt=1-hydroxybenzotriazole; DMF=N,N-dimethylformamide; HOAt=7-aza-1-hydroxybenzotriazole; DCM=dichloromethane; DCE=1,2-dichloroethane; TFA=fluoroacetic acid; Ns=p-nitrophenylsulphonyl; Et=ethyl; Me=methyl; RT=room temperature; MTBE=methyl tert-butyl ether; IPA=iso-propanol; PTFE=polytetrafluoroethylene; BOP=benzotriazole-1-yloxy-tris(dimethylamino)phosphonium hexafluorophosphate; PF₆=hexafluorophosphate; and DCC=1,3-dicyclohexylcarbodiimide.

Methods described herein may be used to make compounds that are, as non-limiting examples, described in European patent application published as 0412814 and international patent applications published as: WO 2004/050657, WO 2006/060122, WO 2004/058726 and WO 2006/060127. Many of the reactants and starting materials described in the following reaction schemes may be made using methods described herein or made using techniques described in any one of WO 2004/050657, WO 2006/060122, WO 2004/058726 and WO 2006/060127, purchased from commercial suppliers and/or made using techniques known to a person of skill in the art.

In the following reaction schemes, chiral centers may be shown in a particular orientation. All possible orientations of these chiral centers are within the scope of the present application. Pure R-forms, pure S-forms, substantially pure R-forms, substantially pure S-forms, racemic mixtures and non-racemic mixtures of chiral centers are provided within the scope of methods described herein.

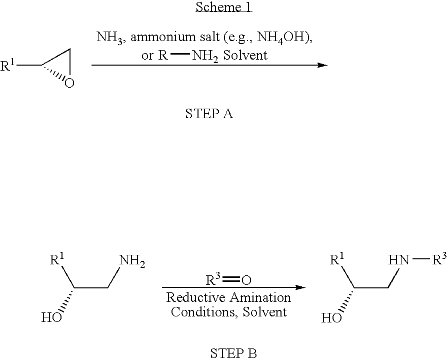

Compounds having a structure of formula (A):

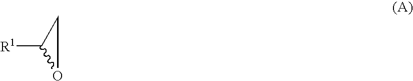

that may be used as a starting material in Scheme 1 may be made using methods described herein, purchased from chemical suppliers and/or are described in European patent application published as 0412814 and international patent applications published as: WO 2004/050657, WO 2006/060122, WO 2004/058726 and WO 2006/060127.

In various embodiments of Scheme 1, R¹ comprises a beta-adrenergic inhibiting moiety or comprises the beta-adrenergic inhibiting moiety when bonded to a

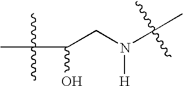

moiety.

In various embodiments of Scheme 1, R¹ may be a moiety of formula (E):

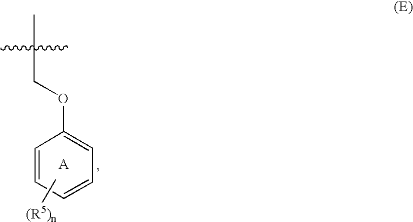

wherein n may be an integer selected from 1, 2, 3, 4 and 5; each R⁵ may be independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₃-C₁₅ heterocycloalkyl, C₃-C₁₅ heterocycloalkenyl, C₃-C₁₅ heterocycloalkynyl, protected C₃-C₁₅ heterocycloalkyl, protected C₃-C₁₅ heterocycloalkenyl, protected C₃-C₁₅ heterocycloalkynyl, C₁-C₁₅ alkoxy and C₃-C₁₅ acylaminoalkyl; each R⁵ may be independently unsubstituted or substituted with one or more heteroatoms selected from the group consisting of oxygen, sulfur, nitrogen and halogen; and each R⁵ has one or more points of attachment to ring A. In various embodiments, n may be 1 and R⁵ may be selected from the group consisting of: a C₆-C₇ heterocycloalkyl having two points of attachment to ring A, halo and cyano.

In various embodiments of Scheme 1, R¹ may be selected from the group consisting of:

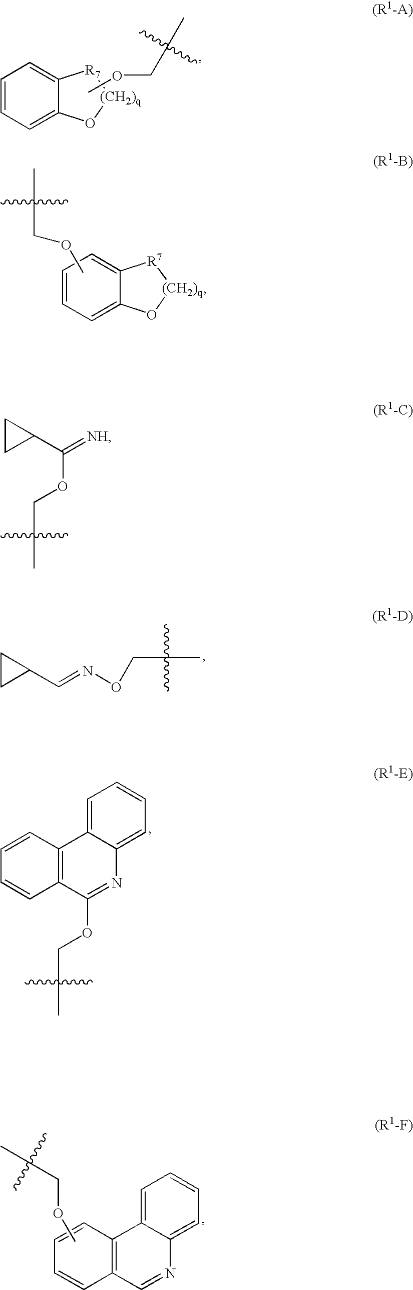

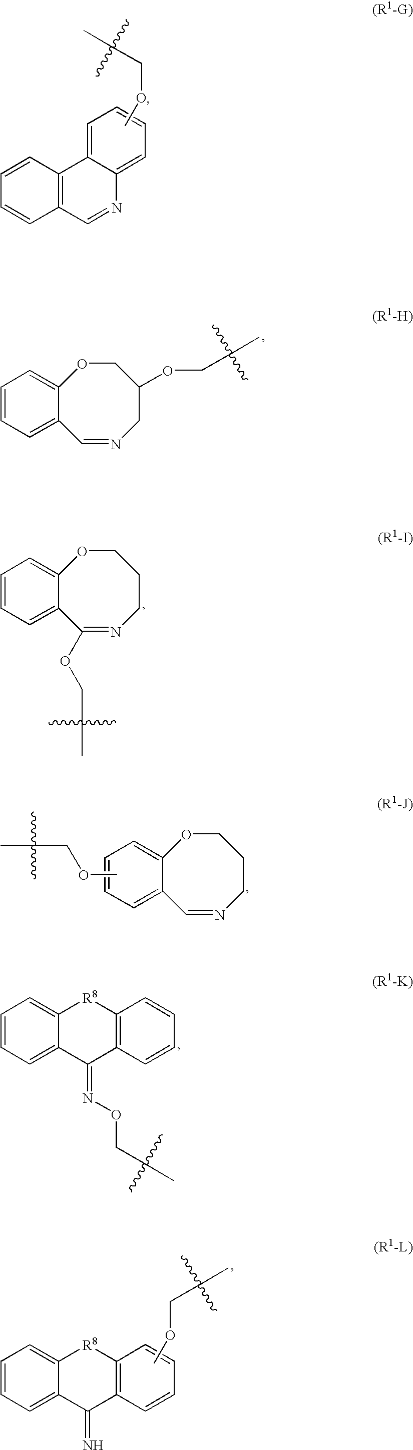

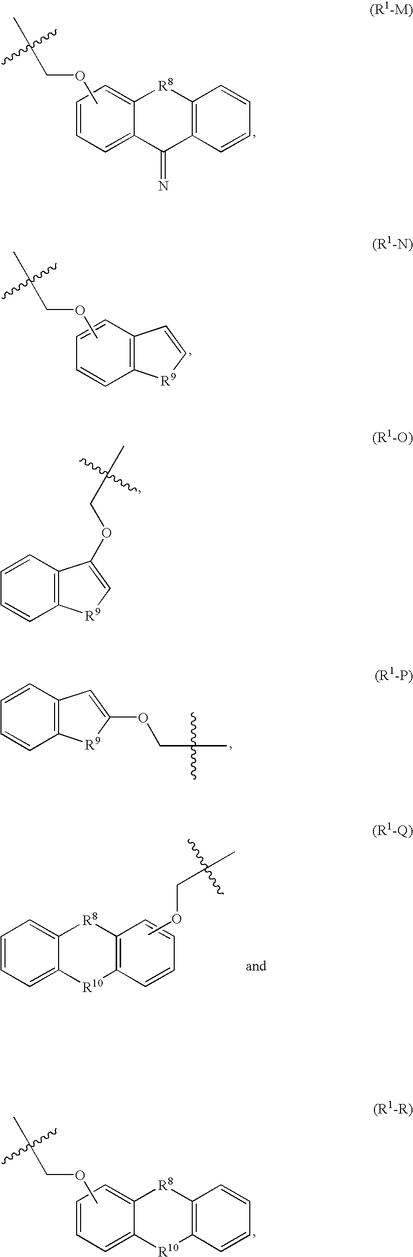

wherein q may be an integer selected from 1, 2 and 3, R⁷ may be O, C═O, S, NH or CH₂, R⁸ may be a bond, —CH₂—, —CH═, —CH₂CH₂—, —CH═CH—, O, S or NH, R⁹ may be —CH₂—, —CH(R⁵)—, —C(R⁵)(R⁵)—, O, S NH or N(R⁵), and R¹⁰ may be a bond or O, where each R⁵ is as defined above.

In various embodiments of Scheme 1, R¹ may be selected from the group consisting of:

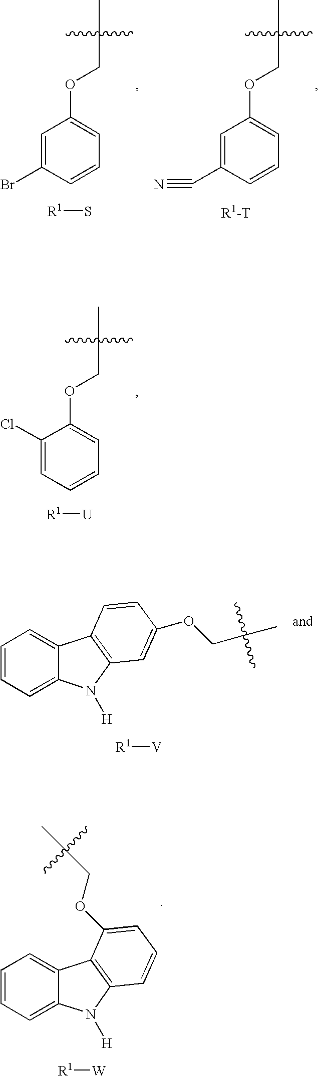

In various embodiments of Scheme 1, step A may be carried out at a temperature of from about 0° C. to about 50° C., from about 10° C. to about 30° C. or at about room temperature. Solvents that may be used in step A comprise or include, without limitation, methanol, water, isopropyl alcohol, gaseous ammonia, or liquid ammonia. Step A may be carried out of a time period of from about 1 hour or more, or from about 10 hours to about 20 hours or about 16 hours. In place of or in addition to NH₃, any of NH₃—NH₄Cl, NH₄, R—NH₂ and/or any ammonium salt may be used, where R is an organic group, such as a protecting group that may removed prior to carrying out step B or may be removed after carrying out step B. NH₃ or NH₄ may be provided in the form of liquid or gaseous ammonia, hydrous or anhydrous ammonia or ammonium salts thereof. In order to reduce byproducts, an excess of NH, NH₃—NH₄Cl, NH₄, R—NH₂ and/or any ammonium salt may be used. R is often a moiety as defined herein by R¹², and is selected independently and denoted R^{12′}. In the case where R^{12′}—NH₂ is used, the product of step A will have a protected amino group (i.e., R^{12′}—NH) in place of NH₂.

The product of step A may be crystallized or may form a crystalline salt, which may provide an opportunity to enhance chemical and/or enantiomeric purity through recrystallization. Isolation of the product of step A may also include an extraction. In some embodiments, a pH selective extraction may provide a substantially pure amine product. Additionally, in the case where R—NH₂ is used in step A, the protecting of step A may be deprotected, before or after any purification and/or isolation steps.

In various embodiments of Scheme 1, R³ comprises the linking moiety and is bonded to the ═O via a carbon atom.

In various embodiments of Scheme 1, R³ may be selected from the group consisting of: C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₃-C₁₅ heterocycloalkyl, C₃-C₁₅ heterocycloalkenyl, C₃-C₁₅ heterocycloalkynyl, protected C₃-C₁₅ heterocycloalkyl, protected C₃-C₁₅ heterocycloalkenyl, protected C₃-C₁₅ heterocycloalkynyl, C₁-C₁₅ alkoxy and C₃-C₁₅ acylaminoalkyl; and R³ may be unsubstituted or substituted with one or more heteroatoms selected from the group consisting of oxygen, sulfur, nitrogen and halogen.

In various embodiments of Scheme 1, R³ may be selected from the group consisting of: substituted C₃-C₁₅ heterocycloalkyl, substituted protected C₃-C₁₅ heterocycloalkyl, substituted C₃-C₁₅ cycloalkyl, substituted protected C₃-C₁₅ cycloalkyl, substituted C₃-C₁₅ heterocycloalkenyl, substituted protected C₃-C₁₅ heterocycloalkenyl, substituted C₃-C₁₅ cycloalkenyl and substituted protected C₃-C₁₅ cycloalkenyl.

In various embodiments of Scheme 1, R³ may be a protected C₃-C₁₅ heterocycloalkyl.

In various embodiments of Scheme 1, R³ may be selected from the group consisting of:

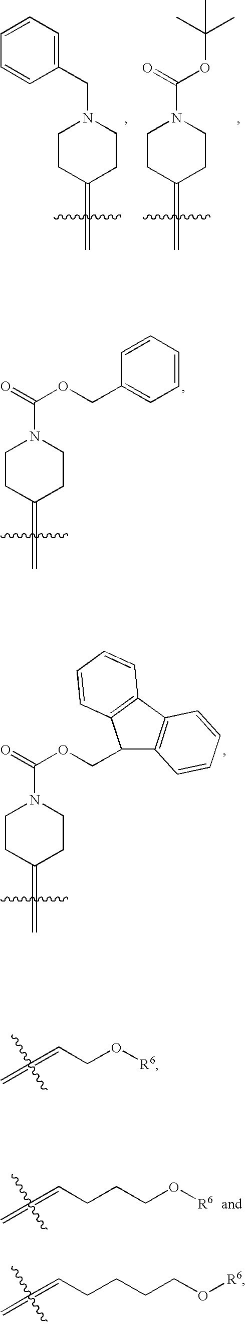

wherein R⁶ comprises a phosphodiesterase inhibiting moiety or comprises a phosphodiesterase inhibiting moiety when bonded to an —O— moiety.

In various embodiments of Scheme 1, R⁶ may be selected from the group consisting of:

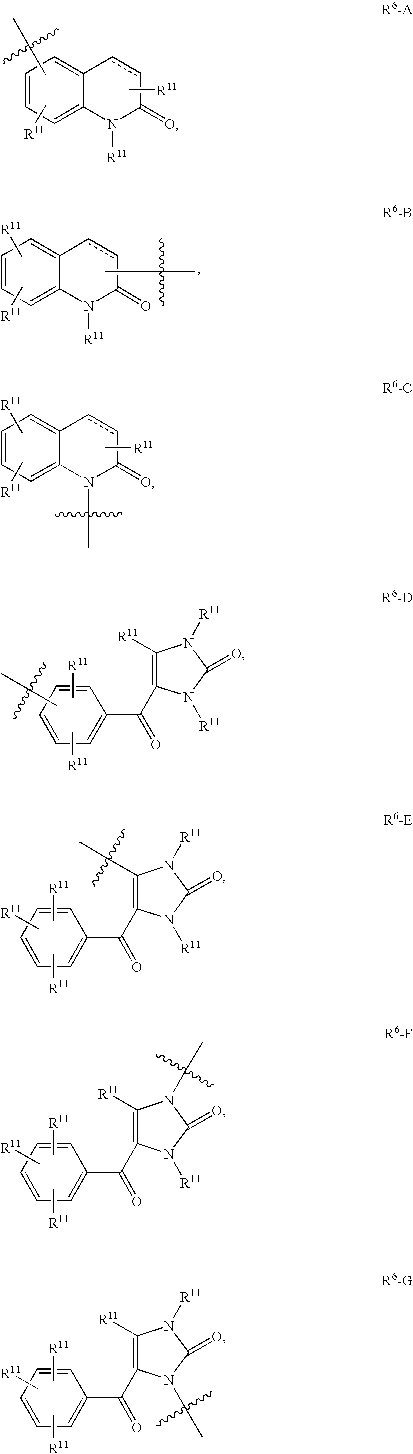

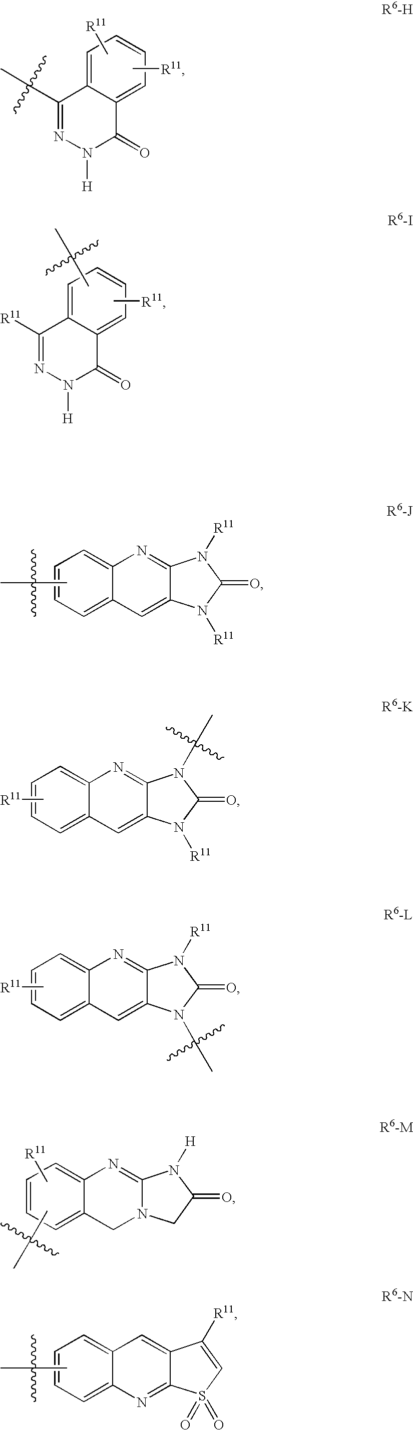

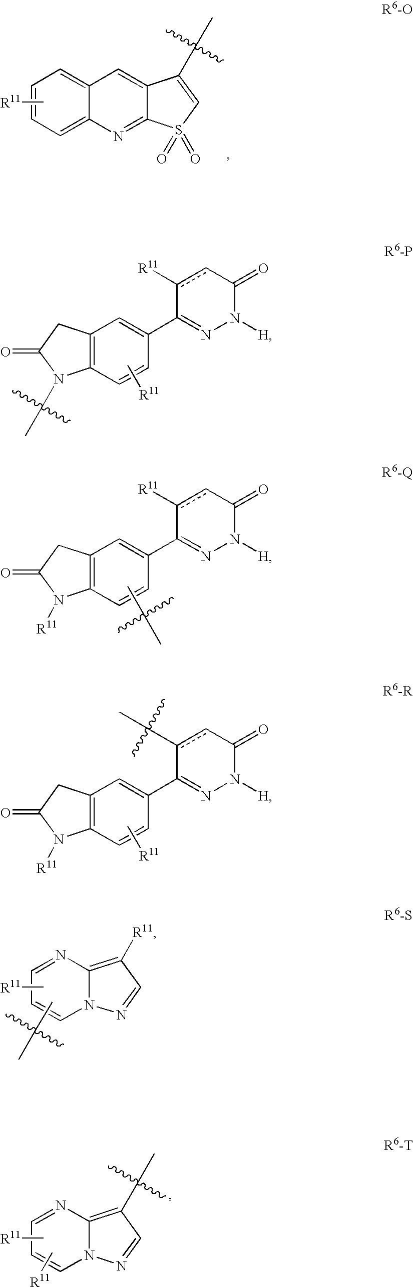

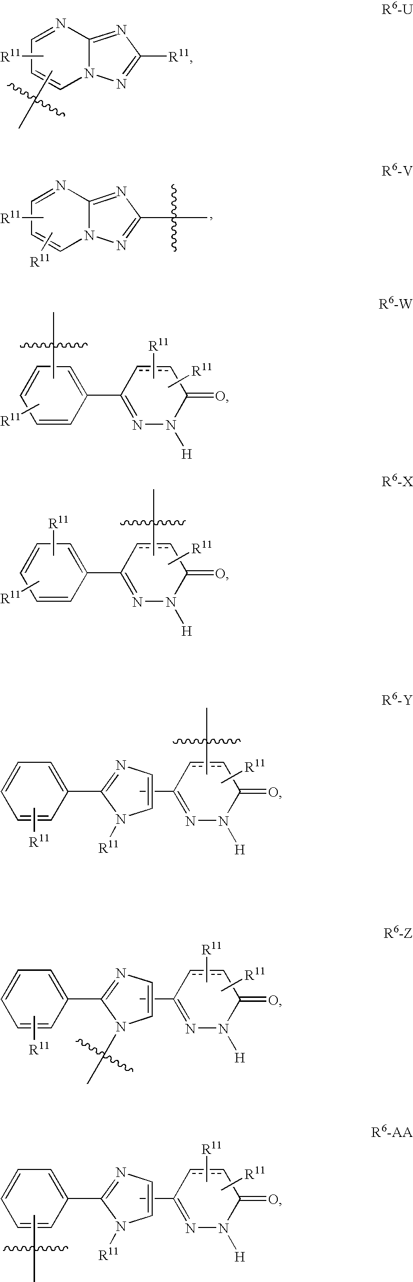

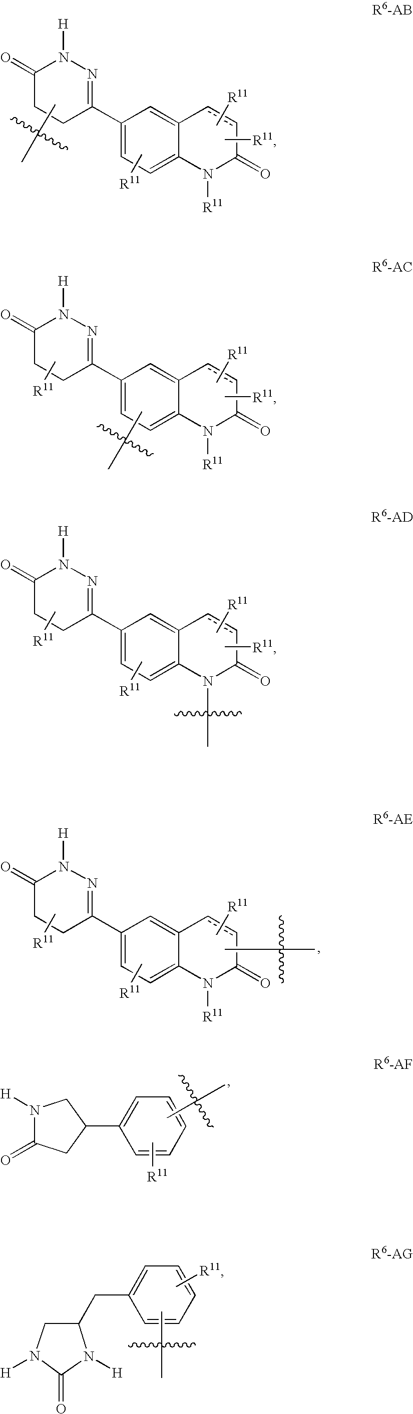

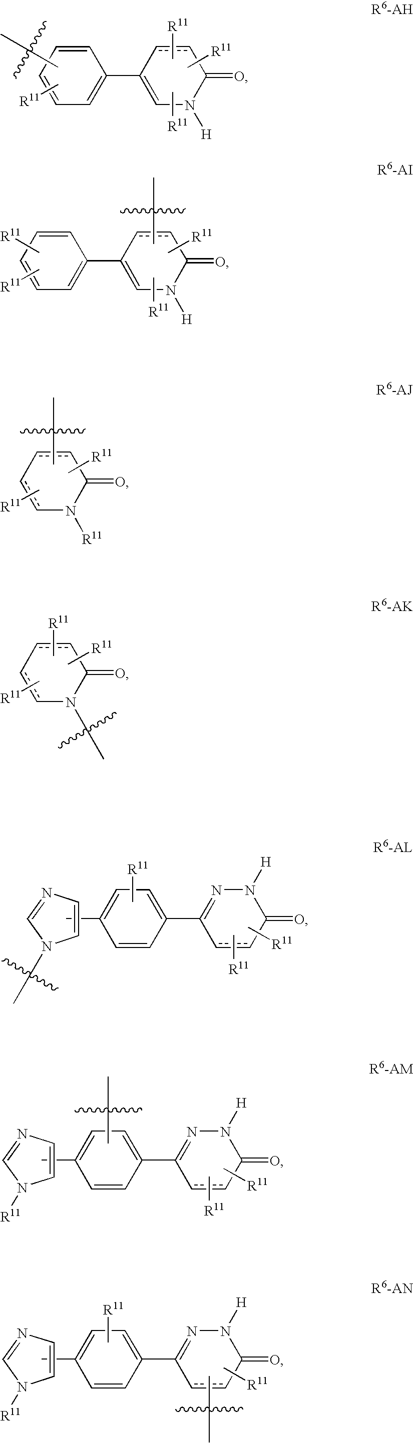

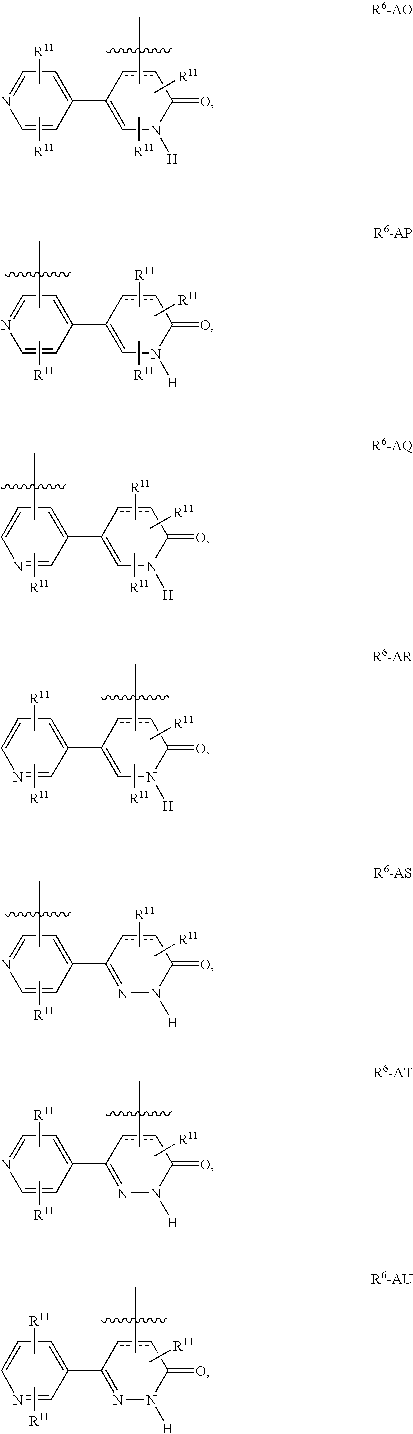

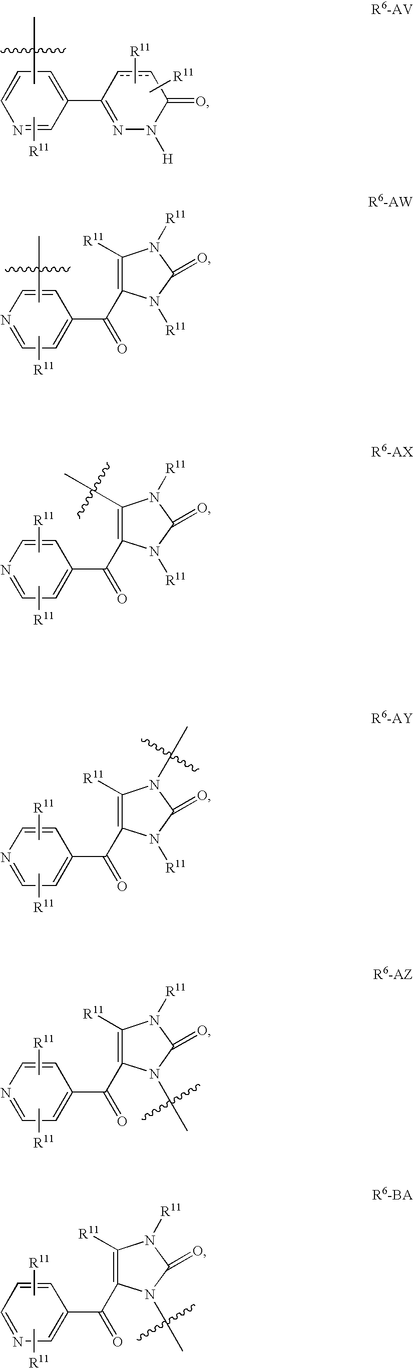

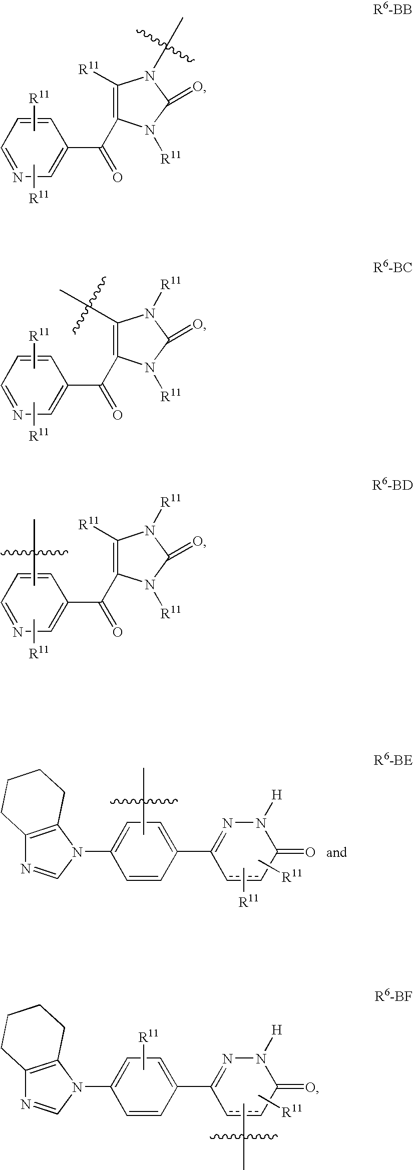

wherein each R¹¹ may be independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₁-C₁₅ alkoxy and C₃-C₁₅ acylaminoalkyl.

In various embodiments of Scheme 1, R⁶ is:

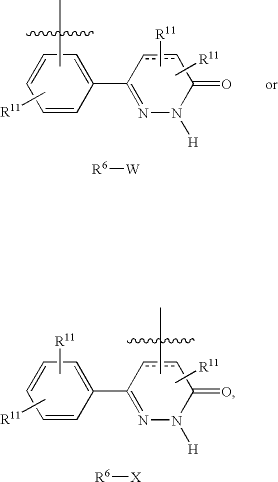

wherein each R¹¹ may be independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₁-C₁₅ alkoxy and C₃-C₁₅ acylaminoalkyl.

In various embodiments of Scheme 1, R⁶ is:

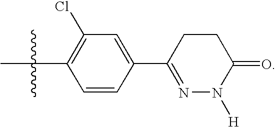

In various embodiments of Scheme 1, R³ is:

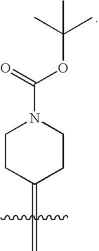

In various embodiments of Scheme 1, step B may be carried out using reductive amination conditions. Examples of such conditions comprise or include without limitation using sodium borohydride and acetic acid in a solvent of dichloromethane (i.e., NaB(OAc)₃H and HOAc in a solvent of CH₂Cl₂). Other reductive amination conditions that are known to a person of skill in the art may also be used (such as NaB(OAc)₃H, NaCNBH₃, LiCNBH₃, NaBH₄/Lewis acid, or Me₄N(OAc)₃BH). Step B may be carried out at a temperature of from about 0° C. to about 50° C., from about 110° C. to about 30° C. or at about room temperature. Step B may be carried out over a period of from about 1 hour or more, from about 10 hours to about 20 hours or for about 16 hours.

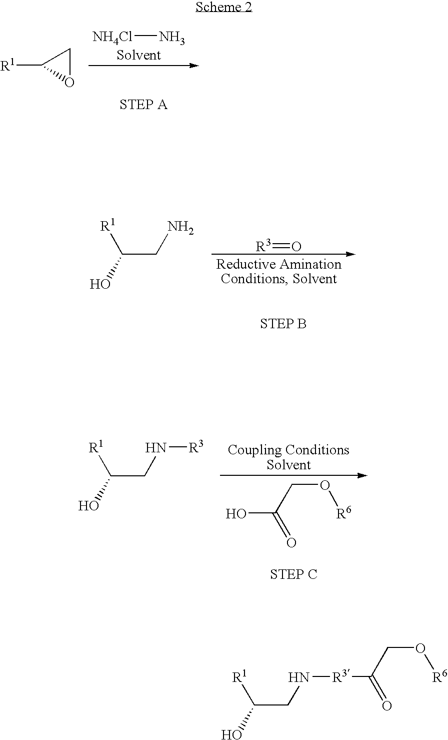

In various embodiments of Scheme 2, the reactants, products, intermediates, substituents thereof and reaction conditions for steps A and B may be as defined for Scheme 1.

In various embodiments of Scheme 2, R^{3′} may be substituted R³, often substituted with

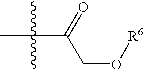

as shown in the product of step C in Scheme 2, wherein R⁶ may be as defined for Scheme 1.

In various embodiments of Scheme 2, step C may be carried out using coupling conditions. Coupling conditions comprise or include, without limitation, standard reagents and conditions used to mediate amide or peptide bond formation. Non-limiting examples comprise or include, BOP PF₆ and DCC. Amide bond formation may be mediate, for example, but not limited to, using acid chloride conditions, or other activated acids. Examples of such conditions comprise or include, but are not limited to, 1-(3-dimethylaminopropyl)-3-ethylcarbodimide hydrochloride (EDCI) and 1-hydroxybenzotriazole (HOBt) using N,N-dimethylformamide (DMF) as a solvent. Alternatively, in place of HOBt, 7-aza-1-hydroxybenzotriazole (HOAt) may be used. Alternative solvents that may be used for step C comprise or include, without limitation, dichlormethane (DCM), 1,2-dichloroethane (DCE), water or a mixture thereof, which mixture may or may not include DMF. Step C may be carried out over a period of from 1 hour or more, from about 10 hours to about 30 hours or for about 20 hours. Step C may be carried out at a temperature of from about 0° C. to about 50° C., from about 110° C. to about 30° C. or at about room temperature. Additional purification of the product may be achieved by using column chromatography on silica gel using an organic mobile phase (e.g., DCM-MeOH) as eluent. Alternatively, column chromatography techniques known to a person of skill in the art may be used.

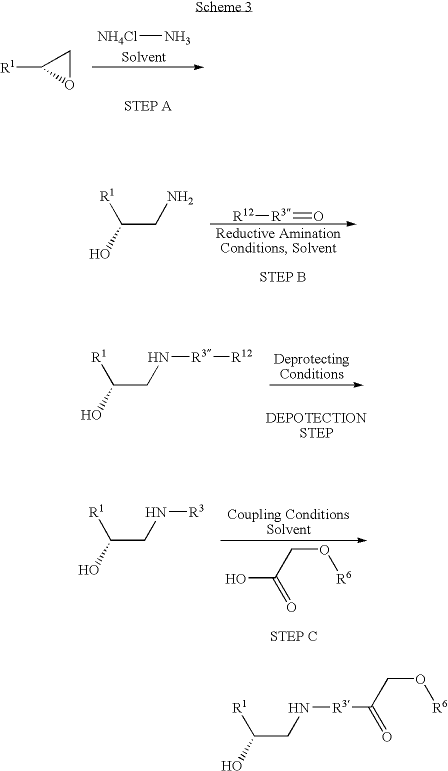

In various embodiments of Scheme 3, the reactants, products, intermediates, substituents thereof and reaction conditions for steps A, B and C may be as defined for Scheme 1 and Scheme 2.

In various embodiments of Scheme 3, R^{3″} may be substituted R³, often substituted with R¹², as shown in Scheme 3, wherein R⁶ may be as defined for Scheme 1 or 2. In some embodiments R^{3″} may have a structure of R³.

In various embodiments of Scheme 3, R^{3″}—R¹² may be selected from the group consisting of: protected C₃-C₁₅ heteroaryl, protected C₁-C₁₅ heteroalkyl, protected C₂-C₁₅ heteroalkenyl, protected C₂-C₁₅ heteroalkynyl, protected C₃-C₁₅ heterocycloalkyl, protected C₃-C₁₅ heterocycloalkenyl, protected C₃-C₁₅ heterocycloalkynyl and protected C₃-C₁₅ acylaminoalkyl; and R³ may be unsubstituted or substituted with one or more heteroatoms selected from the group consisting of oxygen, sulfur, nitrogen and halogen.

In various embodiments of Scheme 3, R^{3″}—R¹² may be selected from the group consisting of: protected C₃-C₁₅ heterocycloalkyl, protected C₃-C₁₅ cycloalkyl, protected C₃-C₁₅ heterocycloalkenyl and protected C₃-C₁₅ cycloalkenyl; and R³ may be unsubstituted or substituted with one or more heteroatoms selected from the group consisting of oxygen, sulfur, nitrogen and halogen.

In various embodiments of Scheme 3, R^{3″}—R¹² may be a protected C₃-C₁₅ heterocycloalkyl.

In various embodiments of Scheme 3, O═R^{3″}—R¹² may be a compound selected from the group consisting of:

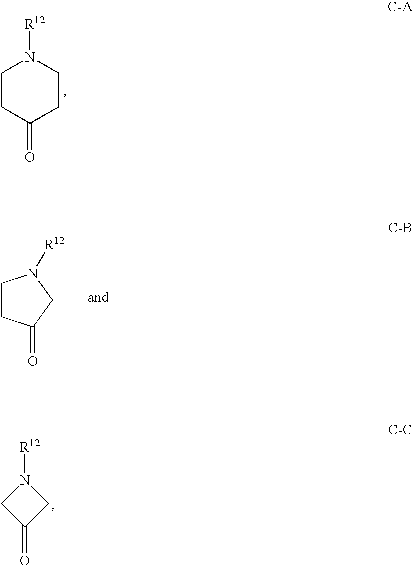

wherein R¹² may be a protecting group.

In various embodiments of Scheme 3, R¹² may be selected from the group consisting of: Boc, Fmoc, Bn and Cbz. R¹² may be selected from a variety of protecting groups that are known to a person of skill in the art. Many such protecting groups are described in the second edition of “Protective Groups in Organic Synthesis”, third edition, by Greene and Wuts (John Wiley & Sons, Inc, 1999) (hereinafter Green and Wuts). Methods of removing protecting groups are also known to a person of skill in the art and are also described in Green and Wuts.

In various embodiments of Scheme 3, R¹² may be a tert-butoxycarbonyl group.

In various embodiments of Scheme 3, R^{3″}—R¹² may be selected from the group consisting of:

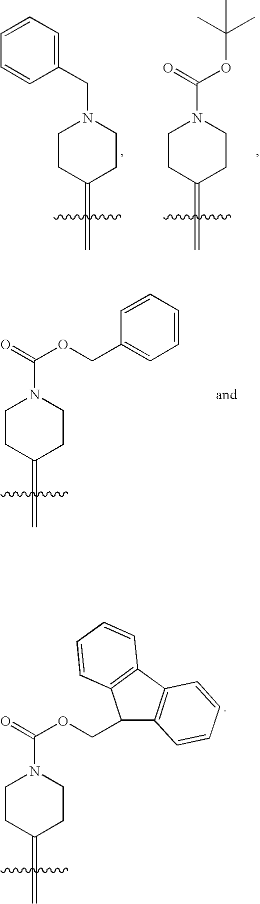

In various embodiments of Scheme 3, R^{3″}—R¹² may be:

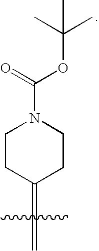

In various embodiments of Scheme 3, the deprotection step may be carried out using acid conditions. The acid conditions may be provided using hydrochloric acid and dioxane. Alternatively, the acid conditions may be provided using HCl and tetrahydrofurane. The HCl may be anhydrous or may be in the form of a solution, for example, but not limited to, 37% HCl in water. Other conditions may include tri-fluoroacetic acid (TFA) in DCM. The deprotection step may be carried out over a period of from about 30 minutes or more, from about 1 hour to about 5 hours or for about 3 hours. The deprotection step may be carried out at a temperature of from about 0° C. to about 50° C., from about 10° C. to about 30° C. or at about room temperature. The acid conditions may be quenched using an alkaline substance, such as NaOH. The product of the deprotection step may be isolated by extraction, for example, using dichloromethane.

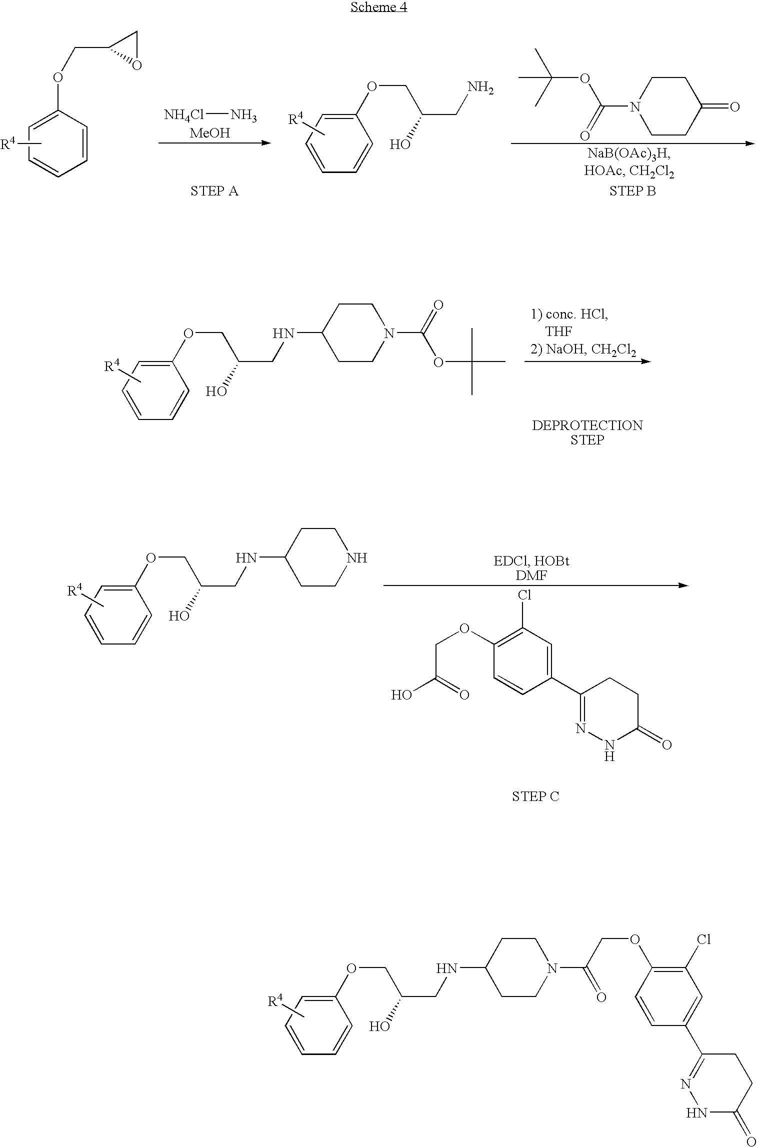

In various embodiments of Scheme 4, the reactants, products, intermediates, substituents thereof and reaction conditions for steps A, B, C and the deprotection step may be as defined for any of Schemes 1, 2 and 3 or as described in Scheme 4.

In various embodiments of Scheme 4, R⁴ may be as defined for R⁵ for any of Schemes 1, 2 and 3.

In various embodiments of Scheme 4, R⁴ may be selected from the group consisting of: Br—(C-3), NC—(C-3), Cl—(C-2),

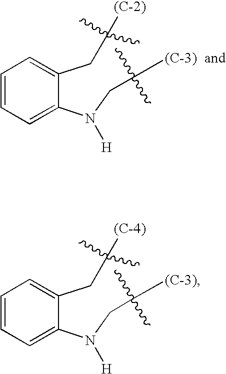

wherein —(C-2) indicates a point of attachment to the carbon at the second position of the benzene ring, —(C-3) indicates a point of attachment to the carbon at the third position of the benzene ring and —(C-4) indicates a point of attachment to the carbon at the fourth position of the benzene ring.

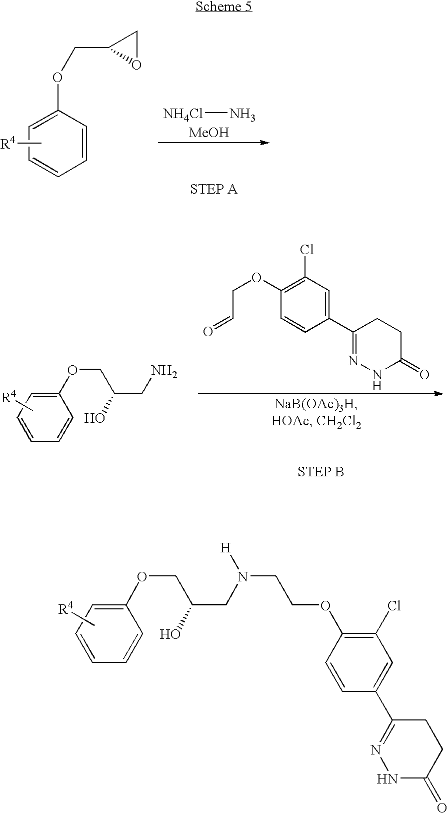

In various embodiments of Scheme 5, the reactants, products, intermediates, substituents thereof and reaction conditions for steps A and B may be as defined for any of Schemes 1, 2, 3 and 4 or as described in Scheme 5.

In various embodiments of Scheme 5, the reactant having the oxo moiety in step B may be made using techniques known to a person of skill in the art and may also be found in WO 2006/060122 and WO 2006/060127.

In various embodiments of Schemes 1, 2, 3, 4 and/or 5 the amine formed by step A may be protected, purified and then deprotected, using protecting groups and conditions and deprotecting conditions known to a person of skill in the art, prior to carrying out step B or after carrying out step B.

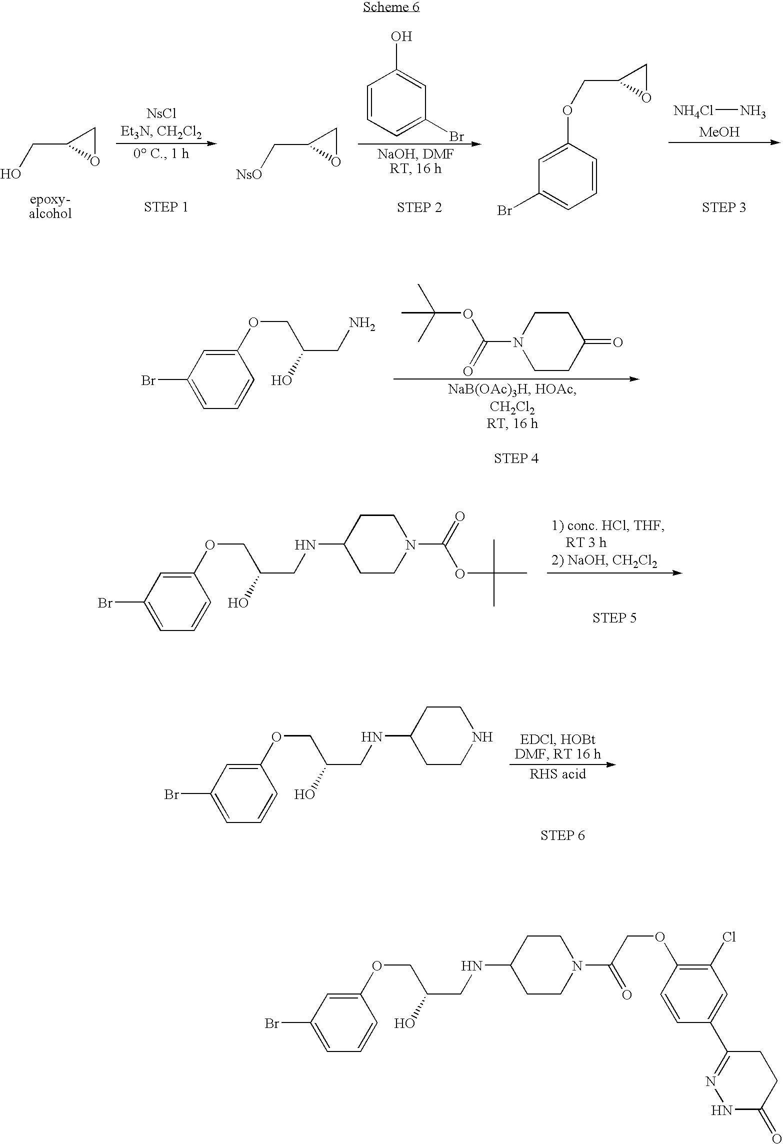

In various embodiments of Scheme 6, RHS acid is the product of Scheme 9 and may be made as described in Scheme 9 or other methods described herein.

In various embodiments of Scheme 6, the reaction conditions may be as described in the Scheme or replaced with the reaction conditions described for steps A, B, C or the deprotection step for Schemes 1, 2, 3, 4 and 5, as appropriate. For example, the reaction conditions for step 3 may be replaced with the reaction conditions for a step A, the reaction conditions for step 4 may be replaced with the reaction conditions for a step B, the reaction conditions for step 5 may be replaced with the reaction conditions for a deprotection step or the reaction conditions for step 6 may be replaced with the reaction conditions for a step C.

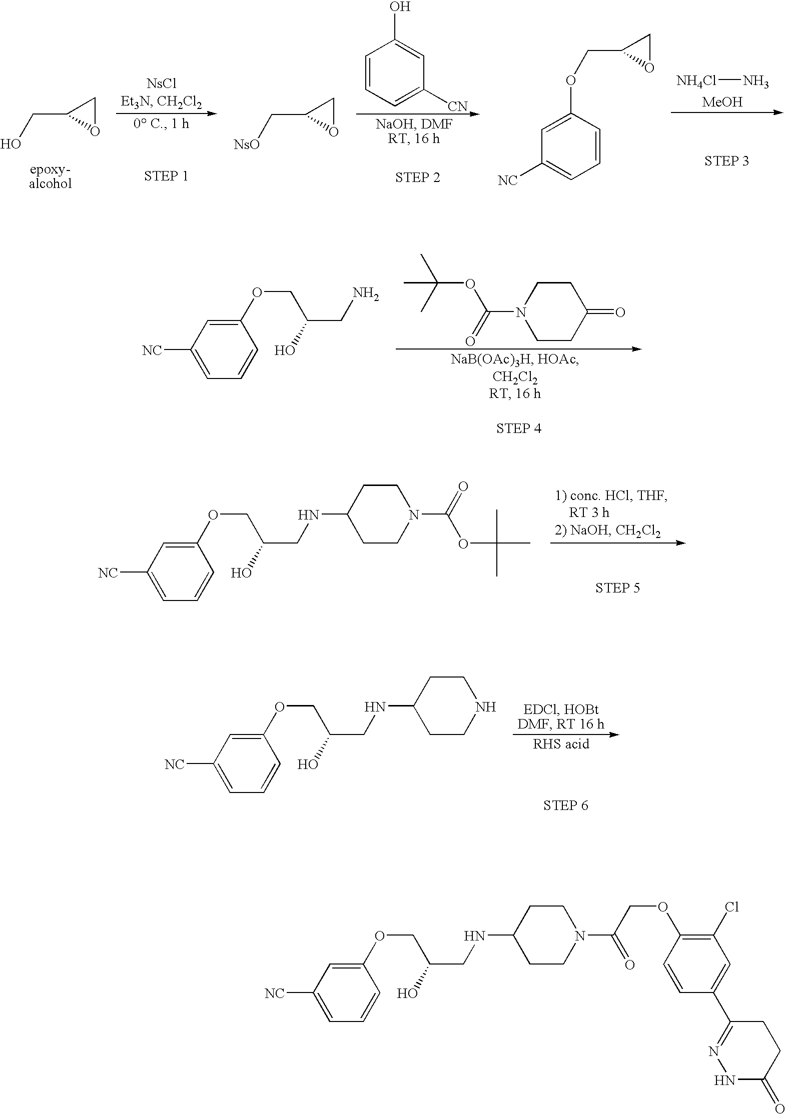

In various embodiments of Scheme 7, RHS acid is the product of Scheme 9 and may be made as described in Scheme 9 or other methods described herein.

In various embodiments of Scheme 7, the reaction conditions may be as described in the Scheme or replaced with the reaction conditions described for steps A, B, C or the deprotection step for Schemes 1, 2, 3, 4 and 5, as appropriate. For example, the reaction conditions for step 3 may be replaced with the reaction conditions for a step A, the reaction conditions for step 4 may be replaced with the reaction conditions for a step B, the reaction conditions for step 5 may be replaced with the reaction conditions for a deprotection step or the reaction conditions for step 6 may be replaced with the reaction conditions for a step C.

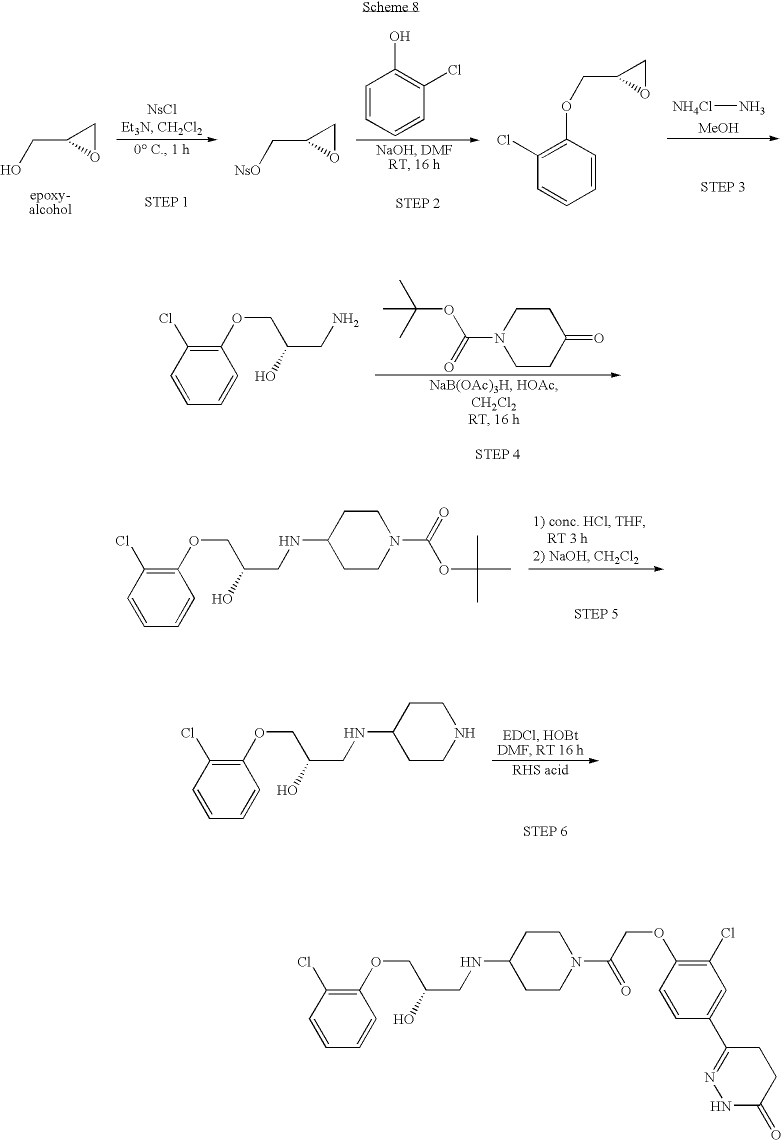

In various embodiments of Scheme 8, RHS acid is the product of Scheme 9 and may be made as described in Scheme 9 or other methods described herein.

In various embodiments of Scheme 8, the reaction conditions may be as described in the Scheme or replaced with the reaction conditions described for steps A, B, C or the deprotection step for Schemes 1, 2, 3, 4 and 5, as appropriate. For example, the reaction conditions for step 3 may be replaced with the reaction conditions for a step A, the reaction conditions for step 4 may be replaced with the reaction conditions for a step B, the reaction conditions for step 5 may be replaced with the reaction conditions for a deprotection step or the reaction conditions for step 6 may be replaced with the reaction conditions for a step C.

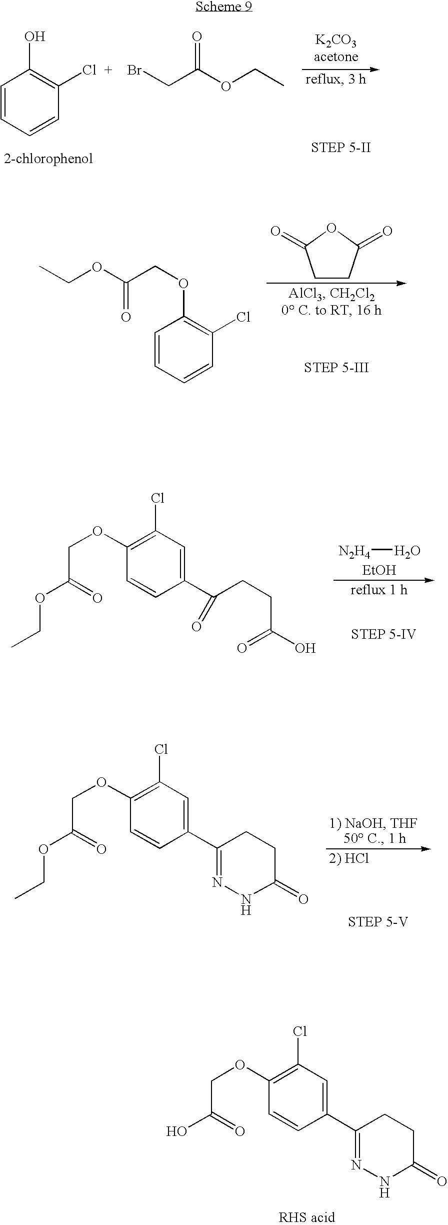

Other methods of making RHS acid are known to a person of skill in the art and may be found, for example, in international patent applications published as: WO 2004/050657, WO 2006/060122, WO 2004/058726 and WO 2006/060127. All four of these international applications are herein incorporated by reference.

### EXAMPLES

### Example 1

### SYNTHESIS OF 4-[3-(2-CHLOROPHENOXY)-2-HYDROXYPROPYLAMINO]PIPERIDINE-1-CARBOXYLIC ACID TERT-BUTYL ESTER (13)

### 3-Nitro-benzenesulfonic acid oxiranylmethyl ester (9)

To a stirred solution of (R)-(+)-glycidol (8, 10.20 g, 138 mmol) and triethyl amine (25 mL, 179 mmol) in DCM (0.3 L) in an ice-water bath was added a solution of 3-nitrobenzenesulfonyl chloride (36.62 g, 165 mmol) in DCM (100 mL) via a dropping funnel. After 30 min, another portion of 3-nitrobenzenesulfonyl chloride (3.0 g, 13.5 mmol) was added and the mixture was stirred for another 2 hours. The reaction was quenched with water (250 mL), and the pH was adjusted to 2 by HCl (1 M). The layers were separated and the aqueous layer was extracted with DCM (150 mL×2). The combined organic layer was washed with saturated NaHCO₃ and dried with anhydrous MgSO₄. The mixture was filtered through a silica pad (2 cm silica on a 600 mL sintered glass funnel) and the filter cake was washed with EtOAc (200 mL). The filtrate was concentrated to dryness by rotary evaporation, and the residue was diluted with ether (1 vol.). Seed crystals were added and the mixture was left in a 5° C. fridge for 2 days. The solid was broken up and triturated with a spatula. The product was isolated by filtration and the filter cake was washed with ether. The crystals were dried by suction and further pumped under high vacuum at room temperature to give 3-nitrobenzenesulfonic acid oxiranylmethyl ester (9) as white crystals, (29.42 g).

### 2-(2-Chloro-phenoxymethyl)-oxirane (10)

To a stirred solution of 2-chlorophenol (6.4 mL, 61 mmol) in DMF (200 mL) was added a solution of NaOH (2.67 g, 66.8 mmol) in minimal amount of water (˜3 mL) drop-wise. The solution was cooled to 0° C. (ice-water bath) and a solution of 3-nitrobenzenesulfonic acid oxiranylmethyl ester (9, 14.39 g, 55.5 mmol) in DMF (50 mL) was added drop-wise over 5 min. DMF (30 mL) was used to rinse the addition funnel. The mixture was removed from the cooling bath and was stirred at room temperature for 21 hours. The reaction mixture was diluted with water (400 mL) and was extracted with MTBE (800 mL in 4 portions). The combined organic was washed with NaHCO₃ (200 mL), NaOH (1 M, 200 mL) and brine (200 mL). After drying with anhydrous MgSO₄, the mixture was filtered through a silica pad (2 cm silica on a 150 mL sintered glass funnel). The filter cake was washed once with ether and the filtrate was concentrated to dryness by rotary evaporation. The solid was triturated with 10% iso-propanol (IPA) in hexanes, filtered, and washed with 5% IPA in hexanes. The crystals were dried by suction and were pumped under high vacuum at room temperature to give 2-(2-chloro-phenoxymethyl)-oxirane (10) as dense crystals with a greenish hue, (7.24 g). Another batch starting from 15.00 g of 9 (57.9 mmol) gave 8.39 g of 10.

### 1-Amino-3-(2-chloro-phenoxy)-propan-2-ol (11)

To a stirred solution of NH₄Cl (8.01 g, 150 mmol) in water (74 mL) and MeOH (300 mL) at 0° C. was introduced NH₃ gas until saturated. Solid 2-(2-chlorophenoxymethyl)oxirane (10, 13.83 g, 74.9 mmol) was added and was rinsed over with MeOH (70 mL). Additional NH₃ was introduced until the system was saturated. The reaction vessel was closed, removed from the cold bath, and the mixture was stirred at room temperature for 16 hours. The mixture was cooled back to 0° C., the vessel was opened, and allowed to warm to room temperature to vent the excess NH₃. The solvent was removed by rotary evaporation and the residue was partitioned between water (200 mL) and ether (200 mL) while maintaining the pH at 7 with HCl (1 M) and/or NaOH (1 M). The organic layer was washed once with water (20 mL, adjust to pH 6˜7 with 1 M HCl) and the combined aqueous layer was extracted with ether (100 mL). The aqueous layer was saturated with NaCl, adjusted to pH 13 with NaOH (6 M), and extracted with DCM (800 mL in 6 portions). The combined DCM extracts were concentrated to near-dryness until most material had crystallized. The solid was triturated with 10% ether in hexanes, filtered, and washed with 10% ether in hexanes. The solid was dried by suction and further pumped under high vacuum at room temperature to give 1-amino-3-(2-chlorophenoxy)propan-2-ol (11) as a white powder (11.74 g).

¹H NMR (CDCl₃, delta): 7.37 (1H, dd, J₁=8.0, J₂=1.6 Hz), 7.22 (1H, m), 6.98-6.90 (2H, m), 4.10-3.97 (3H, m), 3.02 (1H, dd, J₁=12.8, J₂=4.0), 2.94 (1H, J₁=12.8, J₂=6.0), 2.00 (3H, s, br) ppm;

¹³C NMR (CDCl₃, delta): 154.36 (+), 130.47 (−), 127.99 (−), 123.23 (+), 122.06 (−), 113.92 (−), 71.72 (+), 70.23 (−), 44.14 (+) ppm;

### 4-[3-(2-Chlorophenoxy)-2-hydroxypropylamino]piperidine-1-carboxylic acid tert-butyl ester (13)

To a stirred solution of 1-amino-3-(2-chlorophenoxy)propan-2-ol (11, 10.08 g, 50 mmol) in DCM (300 mL) at room temperature was added HOAc (3.0 mL, 50 mmol), NaB(OAc)₃H (13.78 g, 65 mmol), and N-Boc-4-piperidone (12, 10.46 g, 52.5 mol) sequentially. The white suspension was stirred at room temperature under N₂ for 23 hours. The reaction was quenched with a mixture of NaOH (1 M, 100 mL) and brine (100 mL), and was stirred at room temperature for 0.5 hours. The layers were separated, and the aqueous was extracted with DCM (100 mL×2). The combined DCM layer was concentrated to dryness to give 4-[3-(2-chlorophenoxy)-2-hydroxypropylamino]-piperidine-1-carboxylic acid tert-butyl ester (13) as a thick oil.

### Example 2

### SYNTHESIS OF 1-(2-CHLORO-PHENOXY)-3-(PIPERIDIN-4-YLAMINO)—PROPAN-2-OL (14)

To a stirred solution of 4-[3-(2-chlorophenoxy)-2-hydroxypropylamino]piperidine-1-carboxylic acid tert-butyl ester (13, ˜50 mmol and prepared as described in Example 1) in THF (150 mL) at 0° C. was added HCl (6 M, 100 mL). The mixture was stirred at room temperature for 2 hours, then concentrated HCl (100 mL) was added and the reaction was stirred for another 1.5 hours. The mixture was cooled to 0° C. and was basified to pH 6 with NaOH (6 M). The mixture was extracted with DCM (250 mL×2), and the organic was discarded. The aqueous was saturated with NaCl, basified to pH>11 with NaOH (6 M) and was extracted with DCM (1.2 L in 5 portions). The combined extract was concentrated to ˜100 mL and was filtered through a 0.2 micrometer PTFE syringe filter. The filtrate was concentrated to dryness and further dried on high vacuum to give 1-(2-chlorophenoxy)-3-(piperidin-4-ylamino)propan-2-ol (14) as thick semi-solid.

### Example 3

### SYNTHESIS OF 6-[3-CHLORO-4-(2-{4-[3-(2-CHLOROPHENOXY)-2-HYDROXYPROPYLAMINO]-PIPERIDIN-1-YL}-2-OXOETHOXY)-PHENYL]-4,5-DIHYDRO-2H-PYRIDAZIN-3-ONE (15)

### (2-Chlorophenoxy)-acetic acid ethyl ester (3)

To a stirred suspension of K₂CO₃ (60.81 g, 0.44 mol) in acetone (0.5 L) at room temperature was added 2-chlorophenol (1, 41.4 mL, 0.4 mol), ethyl bromoacetate (2, 44.3 mL, 0.4 mol), and more acetone (0.3 L). The mixture was heated under N₂ to reflux for 5 hours. After stirring overnight at room temperature, the mixture was filtered and the filter cake was washed with acetone (2×). The filtrate was concentrated by rotary evaporation to give 2-chlorophenoxy acetic acid ethyl ester (3) as yellow oil (88.86 g).

### 4-(3-Chloro-4-ethoxycarbonylmethoxy-phenyl)-4-oxo-butyric acid (5)

To a stirred suspension of AlCl₃ (134 g, 1.0 mol) in DCM (0.35 L) under N₂ was added succinic anhydride (4, 30.02 g, 0.30 mol). The mixture was cooled in an ice-water bath (<5° C. int.) and 2-chlorophenoxy acetic acid ethyl ester (3, 53.66 g, 0.25 mol) in DCM (70 mL) was added drop-wise via a dropping funnel over 20 min. Stirring was continued in the ice bath for 4 hours, then for 16 hours at room temperature. The mixture was transferred under positive N₂ pressure via a piece of PTFE tubing into a vigorously stirred mixture of ice (˜1 kg) and HCl (3 M, 300 mL). The mixture was extracted with 4:1 DCM-MeOH (total of 4 L in 8 portions). The combined extracts were concentrated by rotary evaporation to near-dryness. The solids were triturated in 1:1 hexane-ether (200 mL), filtered, and the filter cake was washed with 1:1 hexane-ether (2×). The solid was dried by suction and further dried under high vacuum at room temperature to give 4-(3-chloro-4-ethoxycarbonylmethoxyphenyl)-4-oxobutyric acid (5) as white sandy crystals (68.88 g).

¹H NMR (CDCl₃, delta): 8.05 (1H, d, J=2.4 Hz), 7.87 (1H, dd, J=8.4, J₂=2.4 Hz), 6.86 (1H, d, J=8.4 Hz), 4.79 (2H, s), 4.29 (2H, q, J=7.2 Hz), 3.25 (2H, t, J=6.6 Hz), 2.81 (2H, t, J=6.6 Hz), 1.31 (3H, t, J=7.2 Hz) ppm.

### [2-Chloro-4-(6-oxo-1,4,5,6-tetrahydro-pyridazin-3-yl)-phenoxy]-acetic acid ethyl ester (6)

To a stirred suspension of 4-(3-chloro-4-ethoxycarbonylmethoxyphenyl)-4-oxobutyric acid (5, 62.94 g, 0.20 mol) in anhydrous EtOH (0.4 L) at room temperature was added hydrazine hydrate (10.2 mL, 0.21 mol) and the mixture was heated to reflux under N₂ for 3.5 hours. Heating was stopped and seed crystals were added at 50° C. The mixture was diluted with 1:1 hexane-ether (400 mL) at 40° C., and any solid chunk was loosened with a spatula. The crystals were aged in a 4° C. fridge for 1 hour and were filtered. The filter cake was washed with 1:1 hexane-ether and was dried by suction. The material was further dried under high vacuum at room temperature to give [2-Chloro-4-(6-oxo-1,4,5,6-tetrahydro-pyridazin-3-yl)-phenoxy]-acetic acid ethyl ester (6) as white crystals (59.67 g).

¹HNMR (CDCl₃, delta): 8.69 (1H, s), 7.81 (1H, d, J=2.0 Hz), 7.56 (1H, dd, J₁=8.8 Hz, J₂=2.0 Hz), 6.85 (1H, d, J=8.8 Hz), 4.75 (2H, s), 4.28 (2H, q, J=7.2 Hz), 2.95 (2H, t, J=8.0 Hz), 2.61 (2H, t, J=8.0 Hz), 1.31 (3H, t, J=7.2 Hz) ppm.

### [2-Chloro-4-(6-oxo-1,4,5,6-tetrahydro-pyridazin-3-yl)-phenoxy]-acetic acid (7)

To a stirred suspension of [2-Chloro-4-(6-oxo-1,4,5,6-tetrahydropyridazin-3-yl)-phenoxy]acetic acid ethyl ester (6, 58.15 g, 187 mmol) in THF (360 mL) was added NaOH solution (1 M, 560 mL) at room temperature. More THF (200 mL) and water (250 mL) were added and the mixture was heated to 50° C. for 30 mins. The mixture was cooled in a cold water bath and HCl (6 M,˜86 mL) was added to bring the pH to 2 while keeping the solution below 40° C. The solution was further cooled in an ice-water bath with slow agitation for 45 min to allow crystallization. The crystals were filtered and washed with water. Drying by suction overnight and 4 hours under high vacuum at room temperature gave [2-Chloro-4-(6-oxo-1,4,5,6-tetrahydro-pyridazin-3-yl)-phenoxy]-acetic acid (7) as a white crystalline powder (53.88 g).

### 6-[3-Chloro-4-(2-{4-[3-(2-chlorophenoxy)-2-hydroxypropylamino]-piperidin-1-yl}-2-oxoe thoxy)-phenyl]-4,5-dihydro-2H-pyridazin-3-one (15)

To a stirred solution of 1-(2-chlorophenoxy)-3-(piperidin-4-ylamino)propan-2-ol (14, 46 mmol and prepared as described in Example 2) in DMF (100 mL) was added [2-Chloro-4-(6-oxo-1,4,5,6-tetrahydro-pyridazin-3-yl)-phenoxy]acetic acid (7, 14.19 g, 47.3 mmol), 7-hydroxybenzotriazole hydrate (HOBt, 8.39 g, 54.8 mmol), and 1-(3-dimethylaminopropyl)-3-ethylcarbodimide hydrochloride (EDCI, 13.13 g, 68 mmol) sequentially. DMF (150 mL) was used to rinse down each reagent between additions. The resulting solution was stirred at room temperature under N₂ for 20 hours. The reaction was diluted with water (1.6 L) and the pH was raised to 12 with NaOH (6 M). The mixture was extracted with DCM (250 mL×4), and the combined extracts were washed with NaOH (0.1 M, 200 mL) and brine (200 mL). The combined washings were back extracted with DCM (50 mL). The combined DCM extracts were filtered through a celite pad (5 mm celite on a 600 mL sintered glass funnel). The filter cake was washed with a mixture of DCM (200 mL) and MeOH (30 mL). The filtrate was concentrated by rotary evaporation. The residue was purified by column chromatography on silica (Silica H, 800 mL) using a gradient of DCM-MeOH as the eluent: 10:1 (2 L), 7:1 (1.6 L), 5:1 (1.5 L). Fractions that were mostly pure were combined and concentrated. The material was re-purified by column chromatography on silica (230-400 mesh, 800 mL) using a gradient of DCM-MeOH as the eluent: 10:1 (2 L), 5:1 (2 L). Pure fractions were concentrated to dryness and further dried on high vacuum at room temperature for 3 days to give 6-[3-chloro-4-(2-{4-[3-(2-chlorophenoxy)-2-hydroxypropylamino]piperidin-1-yl}-2-oxoet hoxy)phenyl]-4,5-dihydro-2H-pyridazin-3-one (15) as white amorphous foam (16.11 g). The impure fractions were re-purified by column chromatography to give another 5.63 g of product. The final product was then characterized as follows:

### NMR Spectroscopy

¹H NMR spectral data (400 MHz, CDCl₃, δ): 9.36 (br, s, 1H), 7.78 (d, 1H, J=2.0 Hz), 7.50 (dd, 1H, J₁=8.8, J₂=2.4 Hz), 7.33 (dd, 1H, J₁=8.0, J₂=1.2 Hz), 7.22-7.18 (m, 1H), 6.99 (d, 1H, J=8.8), 6.93-6.86 (m, 2H), 4.82 (AB, 1H, J=13.6), 4.78 (AB, 1H, J=13.6), 4.37 (d, 1H, J=13.2), 4.12-3.98 (m, 4H), 3.15 (t, 1H, J=11.6), 2.98-2.69 (m, 6H), 2.89 (t, 2H, J=8.0), 2.56 (t, 2H, J=8.0), 2.04-1.87 (m, 2H), 1.42-1.20 (m, 2H) ppm.

Multiplicity: s=singlet, d=doublet, q=quartet, t=triplet, m=multiplet, br=broad, AB=part of AB quartet.

¹³C NMR Spectral Data (100 MHz, CDCl₃): 167.45, 165.50, 154.54, 154.20, 148.89, 130.38, 129.97, 128.12, 128.01, 125.72, 123.26, 122.98, 122.02, 113.78, 113.18, 72.07, 72.02, 68.60, 68.32, 68.26, 54.78, 48.97, 44.22, 44.18, 41.18, 33.14, 33.09, 32.18, 26.34, 22.44

### Mass Spectrometry

| | |
|---|---|
| MS (ES+): | 549.1 [M + H]⁺ (³⁵Cl, ³⁵Cl) |
| | 551.1 [M + H]⁺ (³⁵Cl, ³⁷Cl) |
| | 571.1 [M + Na]⁺ (³⁵Cl, ³⁵Cl) |
| | 573.1 [M + Na]⁺ (³⁵Cl, ³⁷Cl) |

### Elemental Composition

| | C (%) | H (%) | N (%) | Cl (%) |
|---|---|---|---|---|
| Calculated for | 56.84 | 5.50 | 10.20 | 12.90 |
| C₂₆H₃₀Cl₂N₄O₅ | | | | |
| Found | 55.69 | 5.64 | 9.69 | 13.20 |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims.

What is claimed is:

1. A method of making a compound comprising a beta-adrenergic inhibiting moiety and a linking moiety, the method comprising:
a) reacting a compound of formula (A): 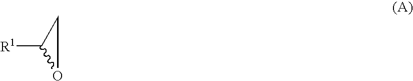
with at least one of NH₃, NH₄, NH₄ClNH₃ and R^{12′}NH₂ to form a compound of formula (B): 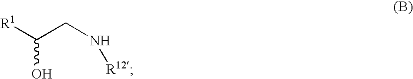
and
b) reacting the compound of formula (B) with a compound of formula (C) under reductive conditions:
R³═O (C),
to form a compound of formula (D): 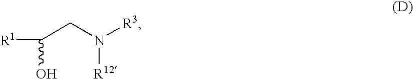
wherein
R¹ comprises the beta-adrenergic inhibiting moiety or comprises the beta-adrenergic inhibiting moiety when bonded to 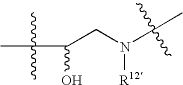
of formula (D);
R³ comprises the linking moiety and is bonded to the ═O of formula (C) via a carbon atom; and
R^{12′} is selected from hydrogen and a protecting group.

2. The method of claim 1 wherein a compound of formula (A) is reacted with an excess of at least one of NH₃, NH₄ and NH₄ClNH₃; and R^{12′} is H.

3. The method of claim 1 wherein a compound of formula (A) is reacted with an excess of NH₃ and R^{12′} is H.

4. The method of claim 1 wherein formula (A) is racemic

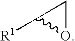

5. The method of claim 1 wherein formula (A) is substantially pure

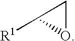

6. The method of claim 1 wherein formula (A) is substantially pure

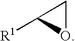

7. The method of claim 1 wherein R³ is selected from the group consisting of: C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₃-C₁₅ heterocycloalkyl, C₃-C₁₅ heterocycloalkenyl, C₃-C₁₅ heterocycloalkynyl, protected C₃-C₁₅ heterocycloalkyl, protected C₃-C₁₅ heterocycloalkenyl, protected C₃-C₁₅ heterocycloalkynyl, C₁-C₁₅ alkoxy and C₃-C₁₅ acylaminoalkyl; and R³ is unsubstituted or substituted with one or more heteroatoms selected from the group consisting of oxygen, sulfur, nitrogen and halogen.

8. The method of claim 1 wherein R³ is selected from the group consisting of: substituted C₃-C₁₅ heterocycloalkyl, substituted protected C₃-C₁₅ heterocycloalkyl, substituted C₃-C₁₅ cycloalkyl, substituted protected C₃-C₁₅ cycloalkyl, substituted
C₃-C₁₅ heterocycloalkenyl, substituted protected C₃-C₁₅ heterocycloalkenyl, substituted C₃-C₁₅ cycloalkenyl and substituted protected C₃-C₁₅ cycloalkenyl.

9. The method of claim 1 wherein R³ is a protected C₃-C₁₅ heterocycloalkyl.

10. The method of claim 1 wherein formula (C) is a compound selected from the group consisting of: 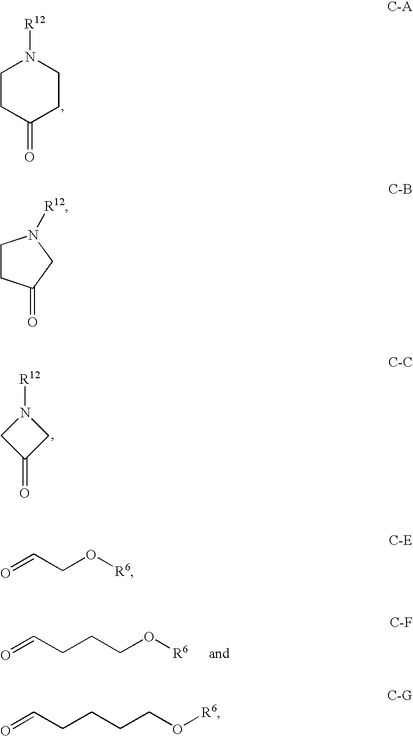
wherein
R⁶ comprises a phosphodiesterase inhibiting moiety or comprises a phosphodiesterase inhibiting moiety when bonded to —O— of any one of formulas C-E, C-F and C-G, and
R¹² is a protecting group.

11. The method of claim 10 wherein R¹² is selected from the group consisting of Boc, Fmoc, Bn and Cbz.

12. The method of claim 10 wherein R⁶ is selected from the group consisting of: 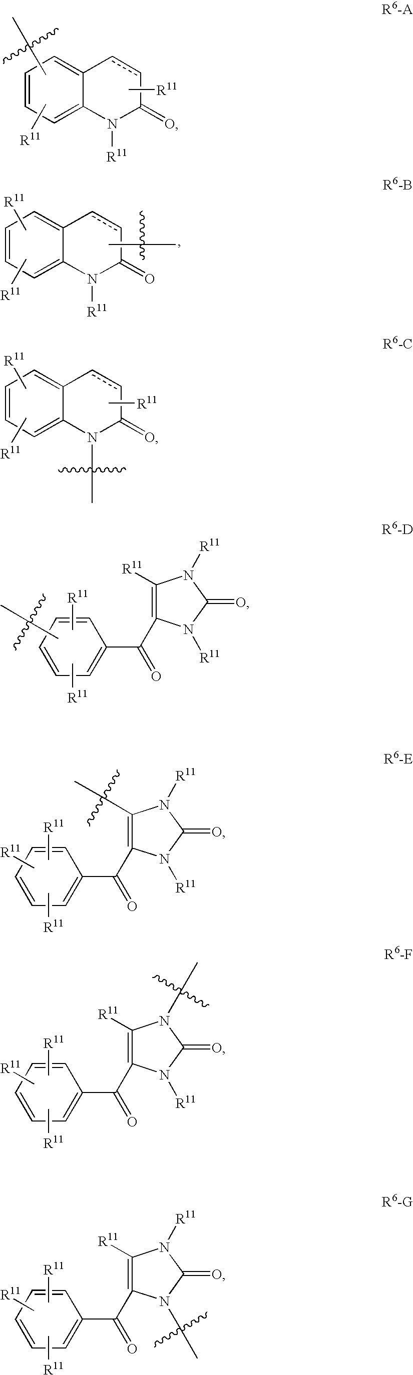 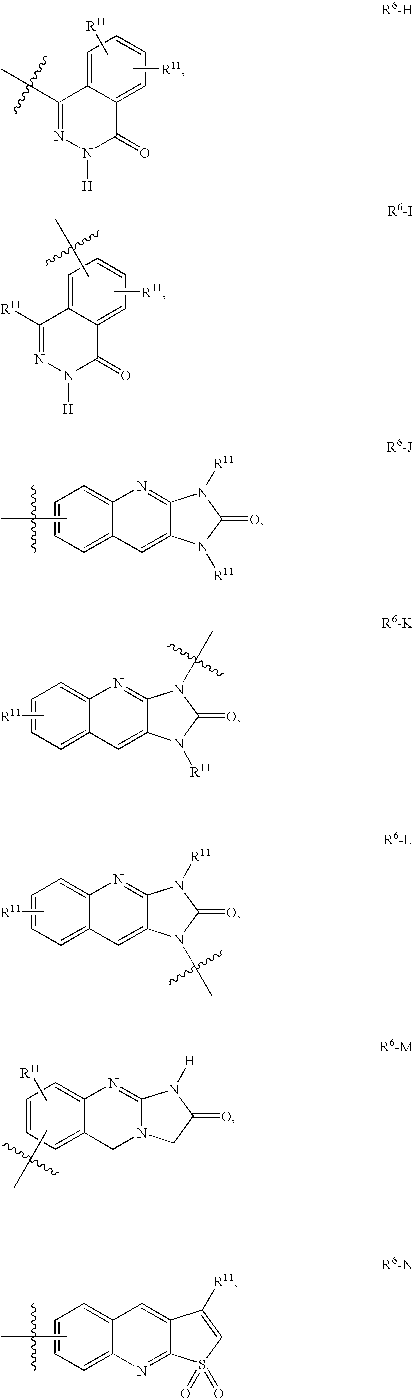 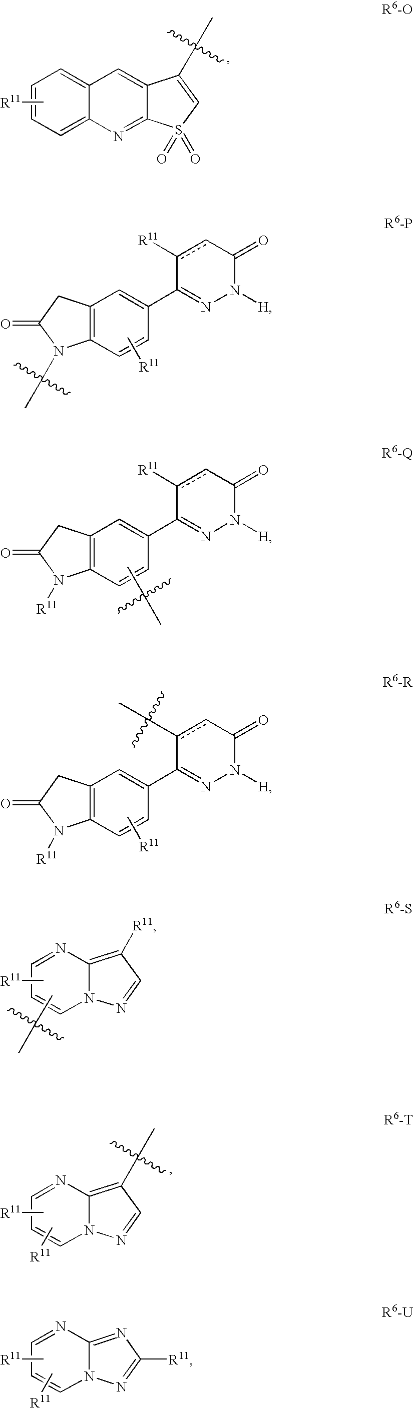 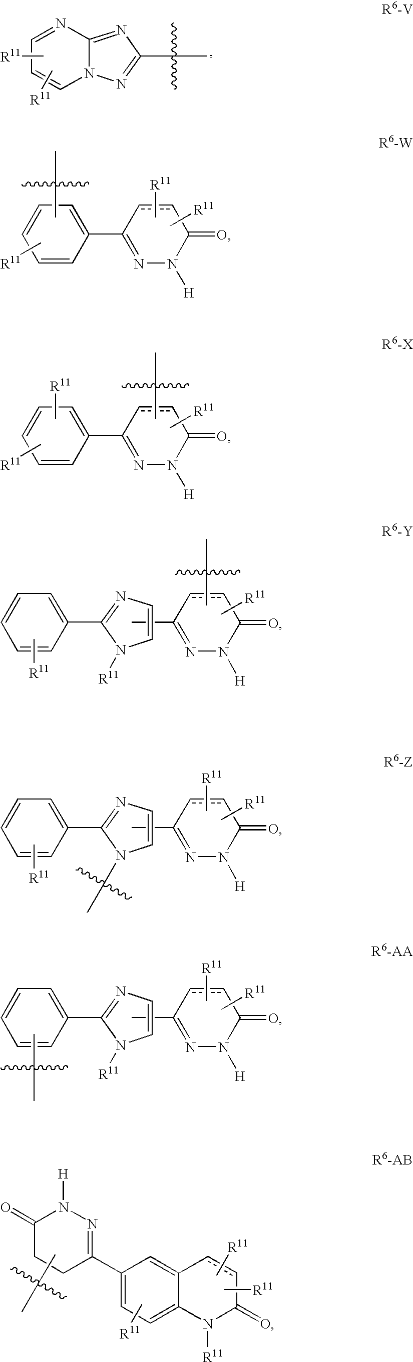 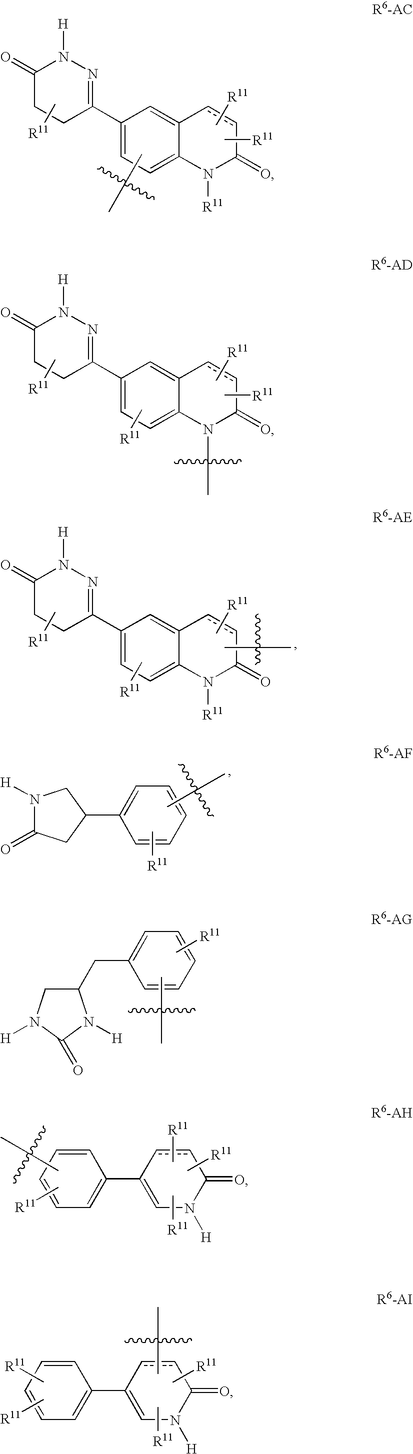 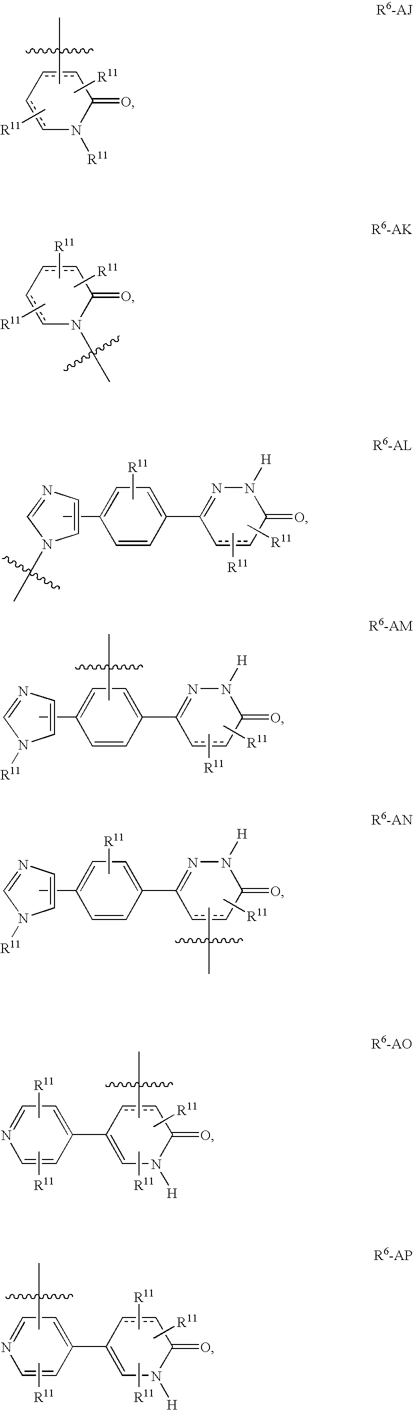 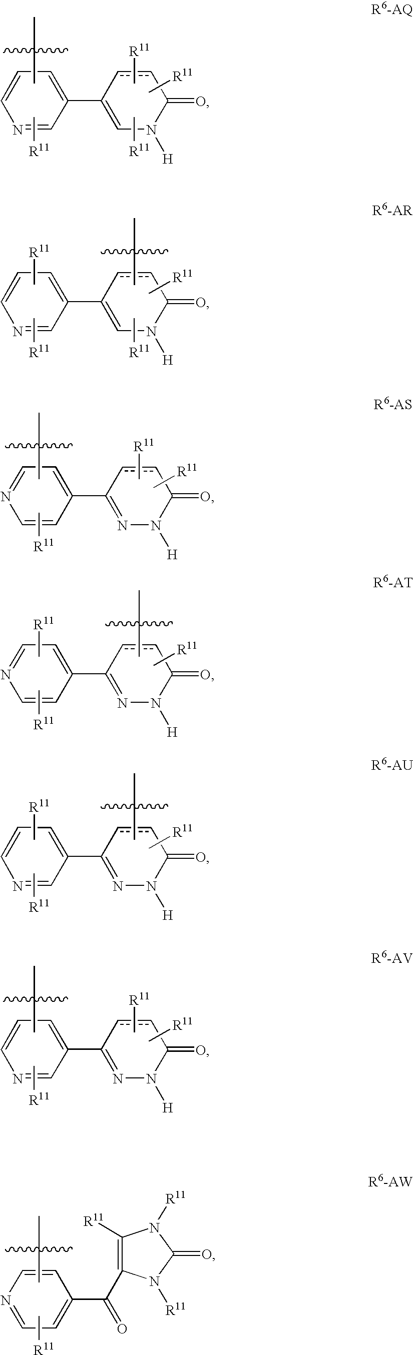 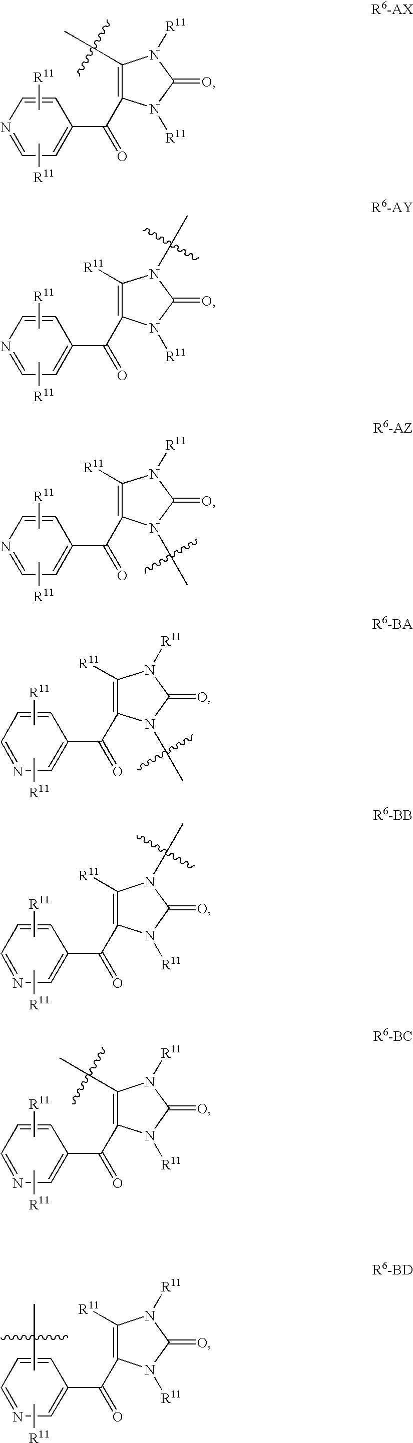 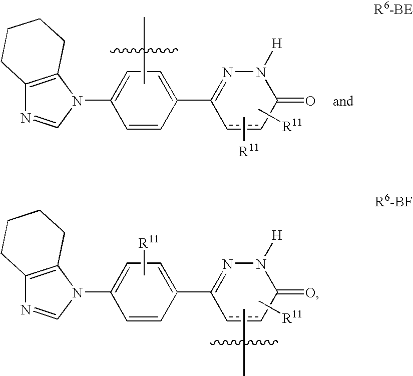
wherein
each R¹¹ is independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₅ heteroalkenyl, C₂-C₁₅ heteroalkynyl, C₃-C₁₅ cycloalkyl, C₃-C₁₅ cycloalkenyl, C₃-C₁₅ cycloalkynyl, C₁-C₁₅ alkoxy and C₃-C₁₅ acylaminoalkyl.

13. The method of claim 10 wherein R⁶ is: 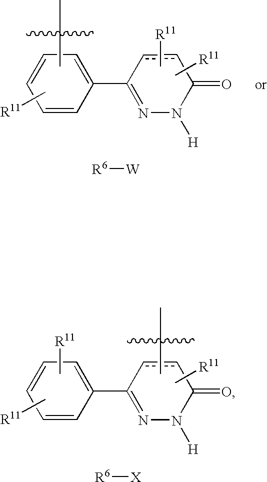
wherein
each R¹¹ is independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₁-C₁₅ alkoxy and C₃-C₁₅ acylaminoalkyl.

14. The method claim 12 wherein each R¹¹ is independently selected from the group consisting of a hydrogen radical and a halo.

15. The method of claim 13 wherein each R¹¹ is independently selected from the group consisting of a hydrogen radical and a halo.

16. The method of claim 10 wherein R⁶ is:

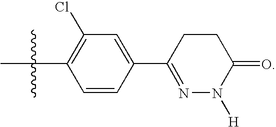

17. The method of claim 1 wherein R³ is selected from the group consisting of:

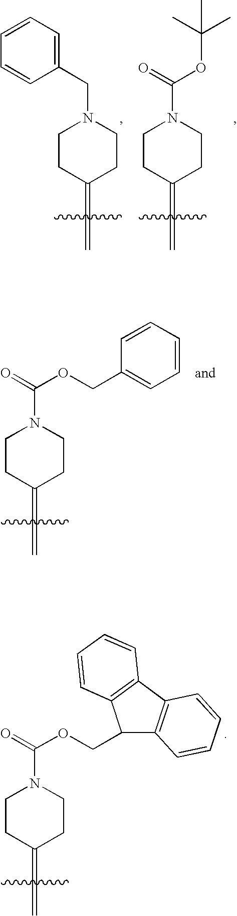

18. The method of claim 1 wherein R¹ is a moiety of formula (E): 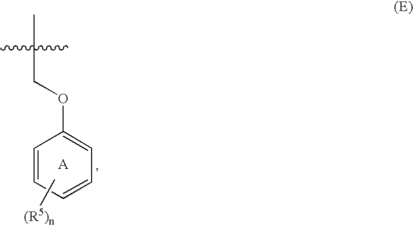
wherein
n is an integer selected from 1, 2, 3, 4 and 5;
each R⁵ is independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₃-C₁₅ heterocycloalkyl, C₃-C₁₅ heterocycloalkenyl, C₃-C₁₅ heterocycloalkynyl, protected C₃-C₁₅ heterocycloalkyl, protected C₃-C₁₅ heterocycloalkenyl, protected C₃-C₁₅ heterocycloalkynyl, C₁-C₁₅ alkoxy and C₃-C₁₅ acylaminoalkyl; each R⁵ is independently unsubstituted or substituted with one or more heteroatoms selected from the group consisting of oxygen, sulfur, nitrogen and halogen; and each R⁵ has one or more points of attachment to ring A.

19. The method of claim 1 wherein R¹ is selected from the group consisting of: 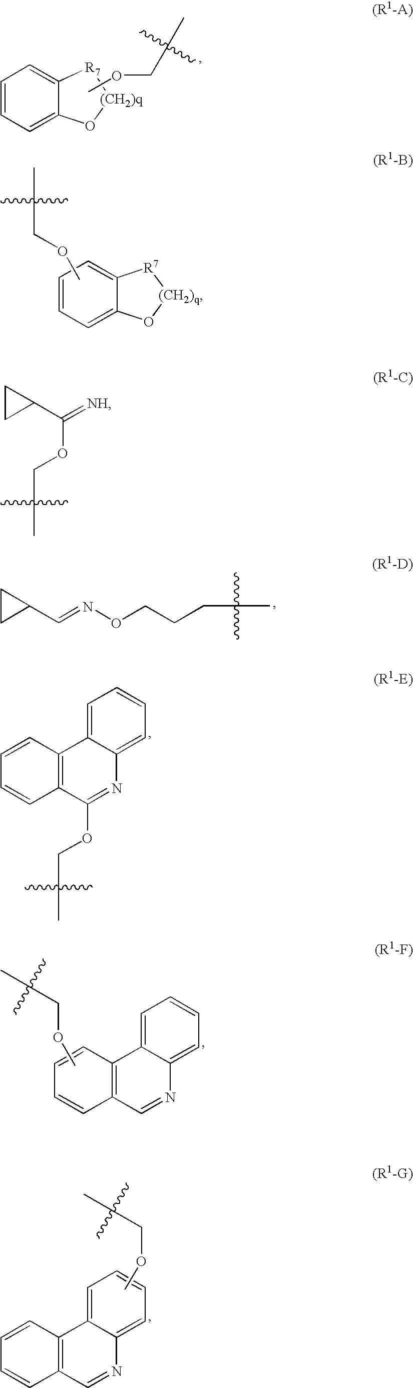 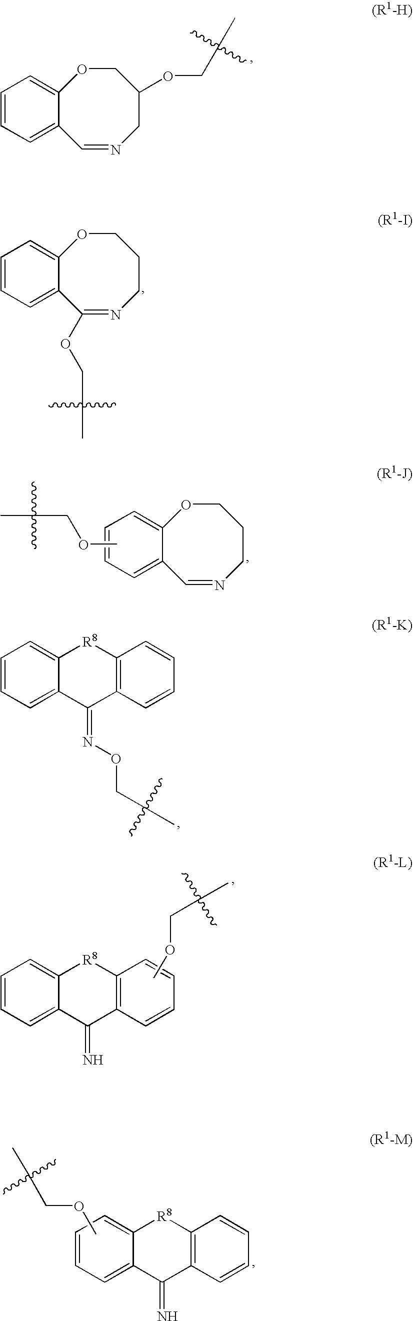 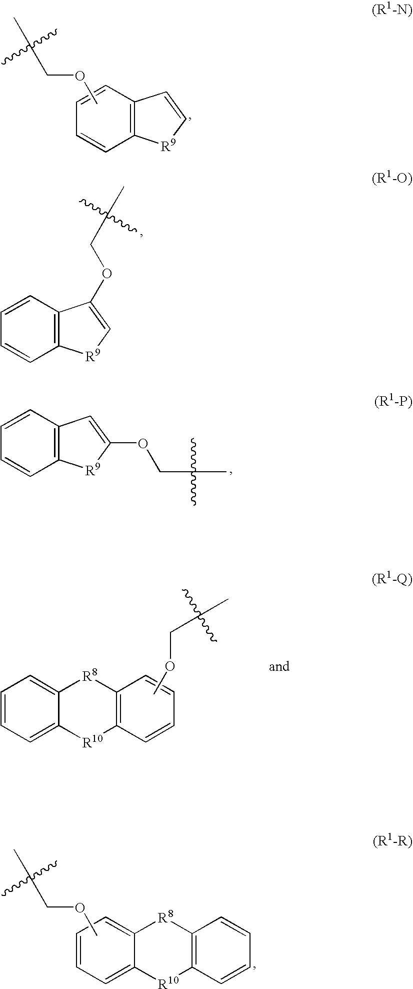
wherein
q is an integer selected from 1, 2 and 3,
R⁷ is O, C═O, S, NH or CH₂,
R⁸ is a bond, H₂C—CH₂, HC═CH, O, S or NH,
R⁹ is O, S or NH and
R¹⁰ is a bond or O.

20. The method of claim 18 wherein n is 1 and R⁵ is selected from the group consisting of a C₅-C₈ heterocycloalkyl having two points of attachment to ring A, halo and cyano.

21. The method of any one of claims 1 to 17 wherein R¹ is selected from the group consisting of:

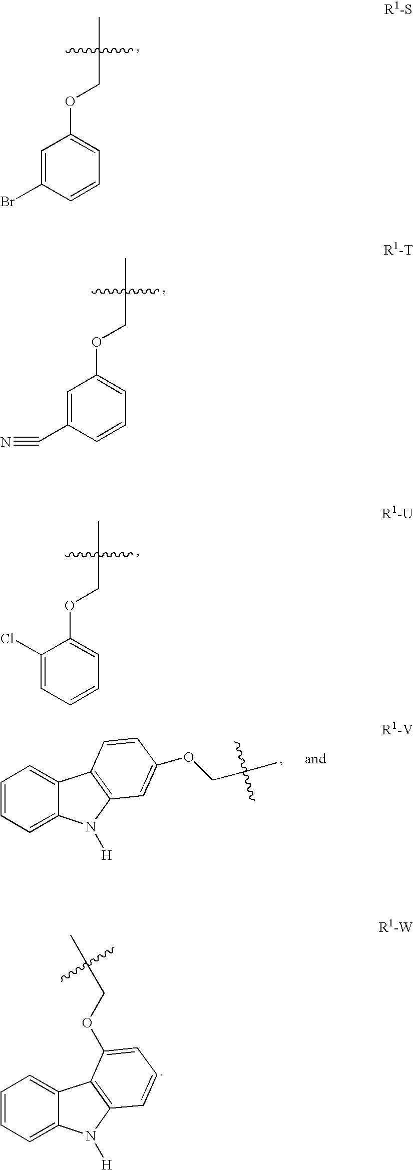

22. A method of making a compound comprising a beta-adrenergic inhibiting moiety and a linking moiety, the method comprising:
a) reacting a compound of formula (A): 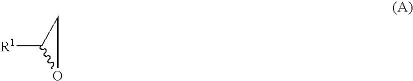
with at least one of NH₃, NH₄, NH₄ClNH₃ and R¹²NH₂ to form a compound of formula (B): 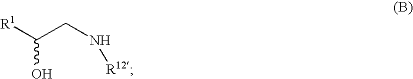
b) reacting the compound of formula (B) with a compound of formula (C):
R³═O (C)
to form a compound of formula (D): 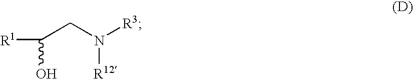
and
c) reacting the compound of formula (D) with a compound of formula (F): 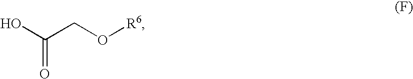
to form a compound of formula (G): 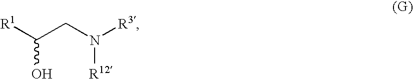
wherein
R¹ comprises the beta-adrenergic inhibiting moiety or comprises the beta-adrenergic inhibiting moiety when bonded to 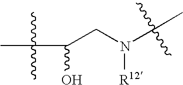
of formula (D);
R³ comprises the linking moiety and is bonded to the ═O in formula (C) via a carbon atom;
R^{3′} is R³ substituted with 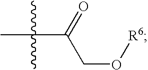
R⁶ comprises a phosphodiesterase inhibiting moiety or comprises a phosphodiesterase inhibiting moiety when bonded to —O— in R^{3′}; and
R^{12′} is selected from H or a protecting group.

23. The method of claim 22 wherein a compound of formula (A) is reacted with an excess of at least one of NH₃, NH₄ and NH₄ClNH₃; and R^{12′} is H.

24. The method of claim 22 wherein a compound of formula (A) is reacted with an excess of NH₃ and R^{12′} is H.

25. The method of claim 22 wherein formula (A) is racemic

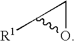

26. The method of claim 22 wherein formula (A) is substantially pure

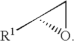

27. The method of claim 22 wherein formula (A) is substantially pure

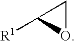

28. The method of claim 22, further comprising removing a protecting group from a compound of formula (D), thereby forming a deprotected compound of formula (D), prior to reacting the deprotected compound of formula (D) with the compound of formula (F), and wherein R12′ is a protecting group.

29. The method of claim 22, further comprising removing a protecting group from a compound of formula (G), thereby forming a deprotected compound of formula (G), and wherein R12′ is a protecting group.

30. The method of claim 22 wherein R³ is selected from the group consisting of: C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₃-C₁₅ heterocycloalkyl, C₃-C₁₅ heterocycloalkenyl, C₃-C₁₅ heterocycloalkynyl, C₁-C₁₅ alkoxy and C₃-C₁₅ acylaminoalkyl; and R³ is unsubstituted or substituted with one or more heteroatoms selected from the group consisting of oxygen, sulfur, nitrogen and halogen.

31. The method of claim 22, further comprising removing a protecting group from a compound of formula (D), thereby forming a deprotected compound of formula (D), prior to reacting the deprotected compound of formula (D) with the compound of formula (F) and wherein R³ is selected from the group consisting of: protected C₃-C₁₅ heteroaryl, protected C₁-C₁₅ heteroalkyl, protected C₂-C₁₅ heteroalkenyl, protected C₂-C₁₅ heteroalkynyl, protected C₃-C₁₅ heterocycloalkyl, protected C₃-C₁₅ heterocycloalkenyl, protected C₃-C₁₅ heterocycloalkynyl and protected C₃-C₁₅ acylaminoalkyl; and R³ is unsubstituted or substituted with one or more heteroatoms selected from the group consisting of oxygen, sulfur, nitrogen and halogen.

32. The method of claim 31 wherein R³ is selected from the group consisting of: protected C₃-C₁₅ heterocycloalkyl, protected C₃-C₁₅ cycloalkyl, protected C₃-C₁₅ heterocycloalkenyl and protected C₃-C₁₅ cycloalkenyl; and R³ is unsubstituted or substituted with one or more heteroatoms selected from the group consisting of oxygen, sulfur, nitrogen and halogen.

33. The method of claim 31 wherein R³ is a protected C₃-C₁₅ heterocycloalkyl.

34. The method claim 31 wherein R³ is selected from the group consisting of:

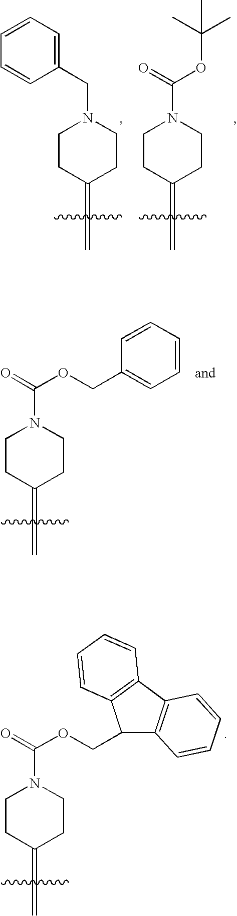

35. The method of claim 31 wherein formula (C) is a compound selected from the group consisting of: 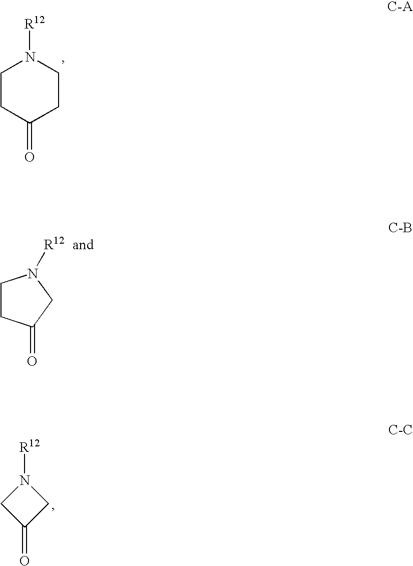
wherein
R¹² is a protecting group.

36. The method of claim 35 wherein R¹² is selected from the group consisting of: Boc, Fmoc, Bn and Cbz.

37. The method of claim 35 wherein the protecting group is a tert-butoxycarbonyl group.

38. The method of claim 31 wherein R³ is:

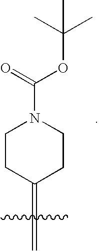

39. The method of claim 22 wherein R⁶ is selected from the group consisting of: 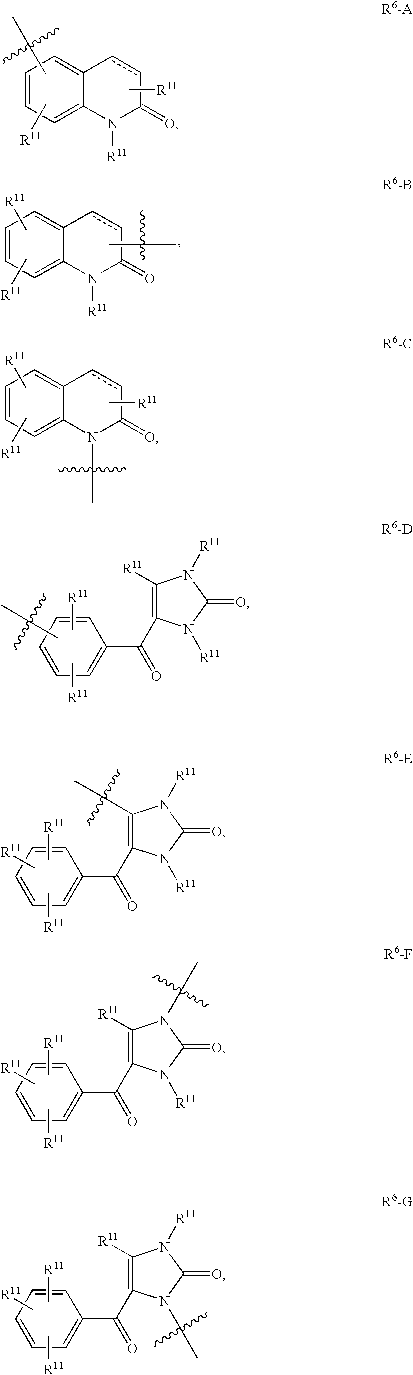 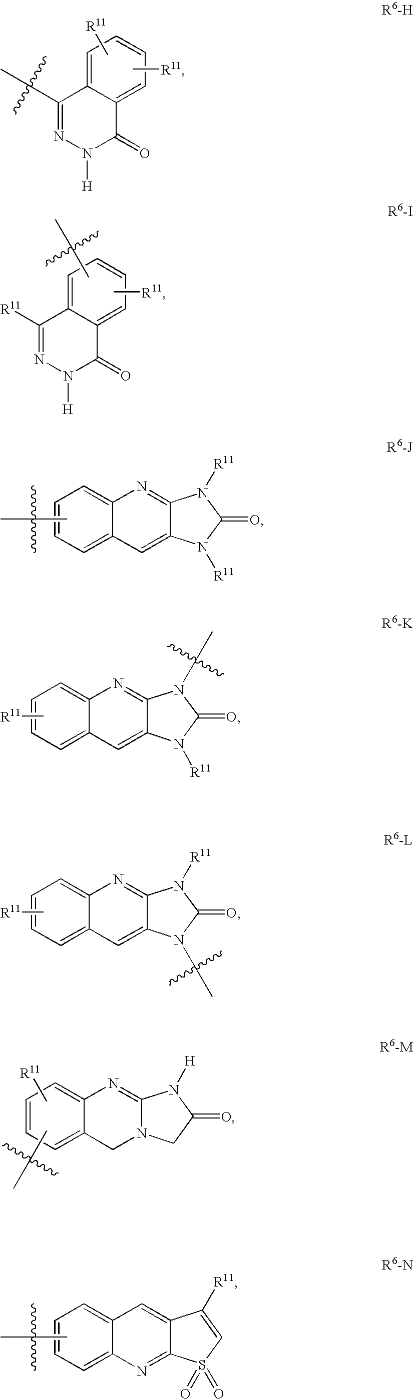 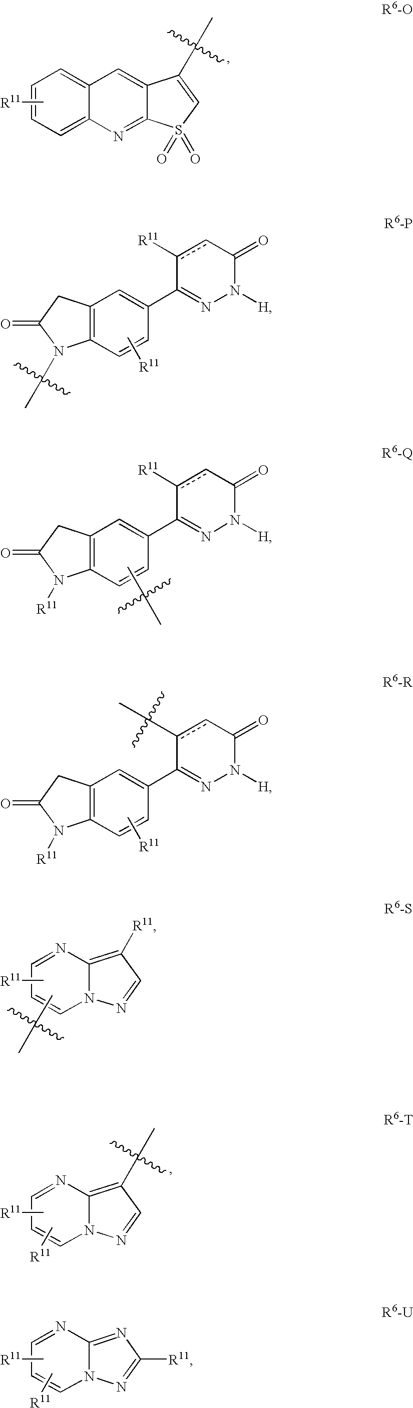 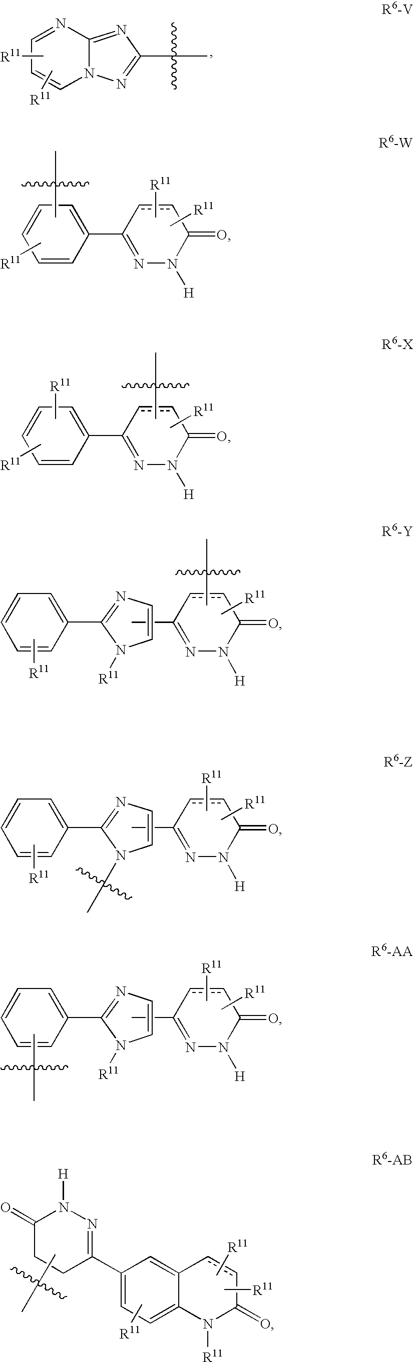 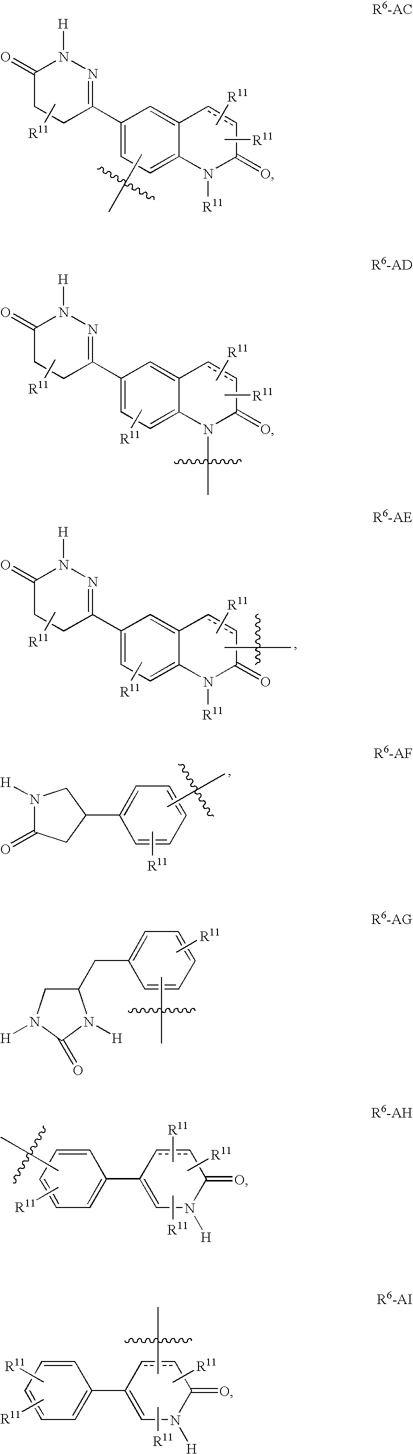 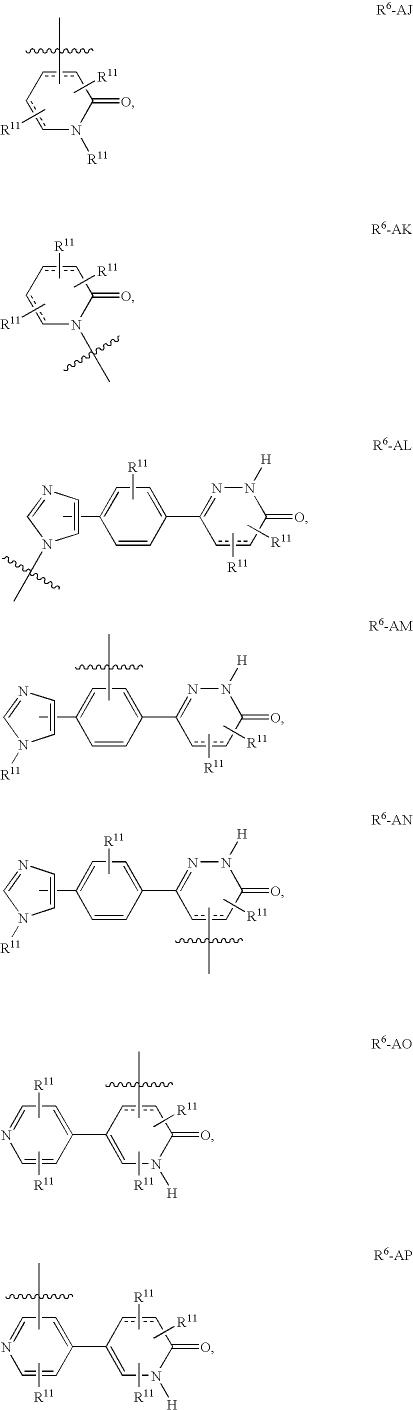 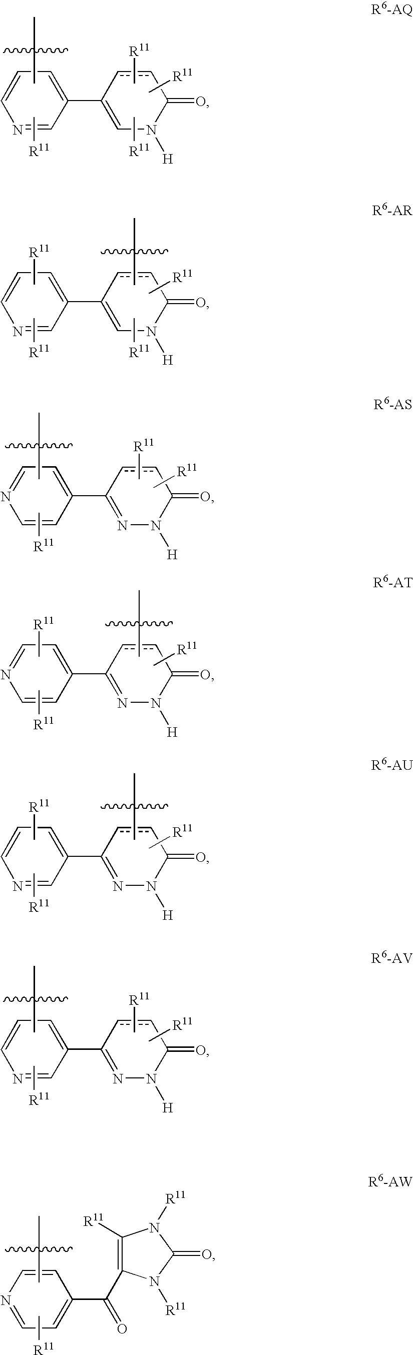 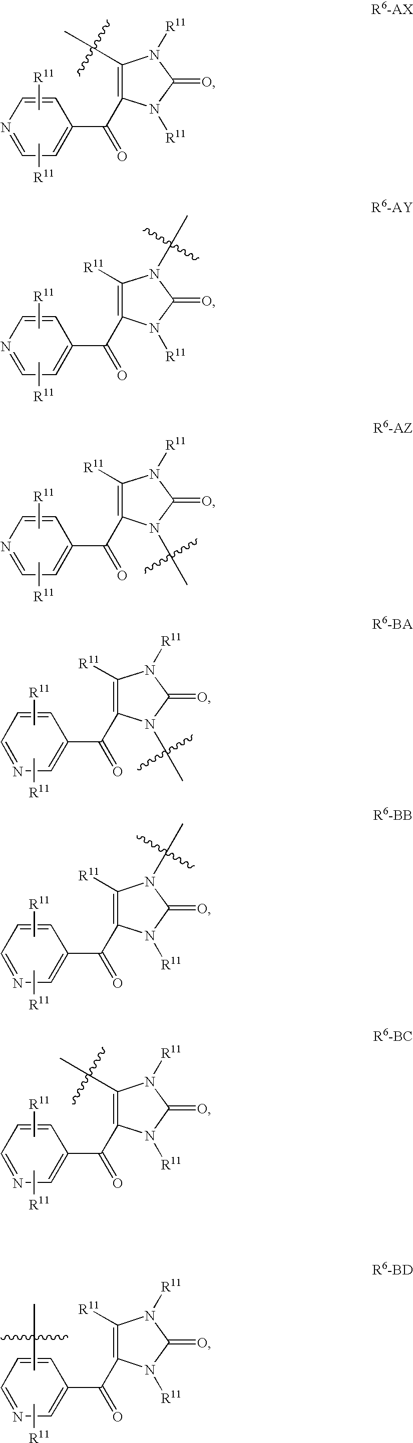 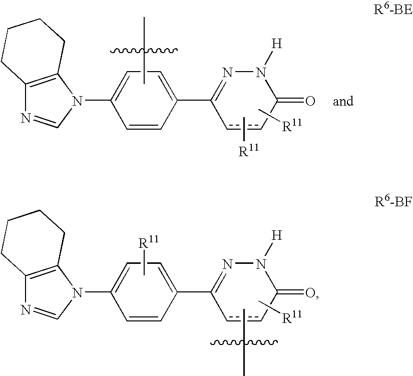
wherein
each R¹¹ is independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₁-C₁₅ alkoxy and C₃-C₁₅ acylaminoalkyl.

40. The method of claim 22 wherein R⁶ is: 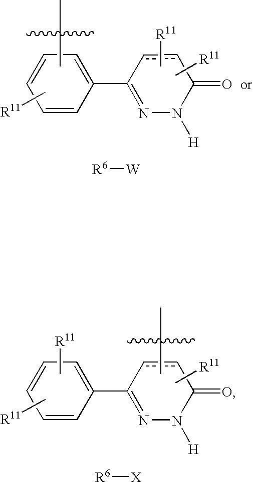
wherein
each R¹¹ is independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₁-C₁₅ alkoxy and C₃-C₁₅ acylaminoalkyl.

41. The method of claim 39 wherein each R¹¹ is independently selected from the group consisting of: a hydrogen radical and a halo.

42. The method of claim 40 wherein each R¹¹ is independently selected from the group consisting of: a hydrogen radical and a halo.

43. The method of claim 22 wherein R⁶ is:

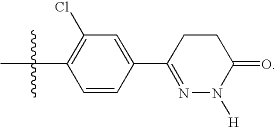

44. The method of claim 22 wherein R¹ is a moiety of formula (E): 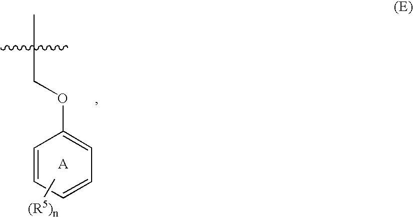
wherein
n is an integer selected from 1, 2, 3, 4 and 5;
each R⁵ is independently selected from the group consisting of: hydrogen radical, halo, cyano, nitro, trifluoromethyl, trifluoromethoxy, C₃-C₁₅ aryl, C₃-C₁₅ heteroaryl, C₁-C₁₅ alkyl, C₂-C₁₅ alkenyl, C₂-C₁₅ alkynyl, C₁-C₁₅ heteroalkyl, C₂-C₁₅ heteroalkenyl, C₂-C₁₅ heteroalkynyl, C₃-C₁₅ cycloalkyl, C₃-C₁₅ cycloalkenyl, C₃-C₁₅ cycloalkynyl, C₃-C₁₅ heterocycloalkyl, C₃-C₁₅ heterocycloalkenyl, C₃-C₁₅ heterocycloalkynyl, protected C₃-C₁₅ heterocycloalkyl, protected C₃-C₁₅ heterocycloalkenyl, protected C₃-C₁₅ heterocycloalkynyl, C₁-C₁₅ alkoxy and C₃-C₁₅ acylaminoalkyl; each R⁵ is independently unsubstituted or substituted with one or more heteroatoms selected from the group consisting of oxygen, sulfur, nitrogen and halogen; and each R⁵ has one or more points of attachment to ring A.

45. The method of claim 22 wherein R¹ is selected from the group consisting of: 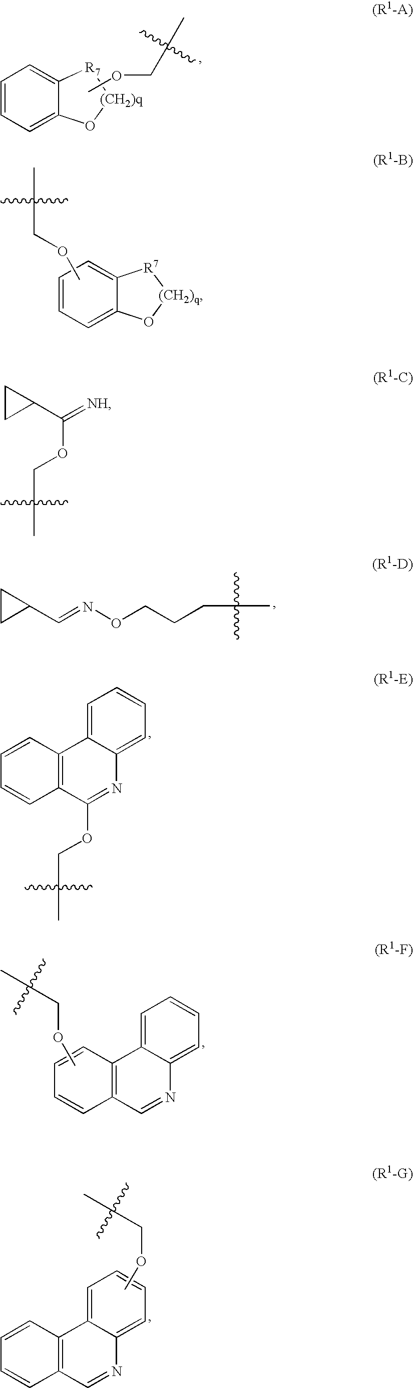 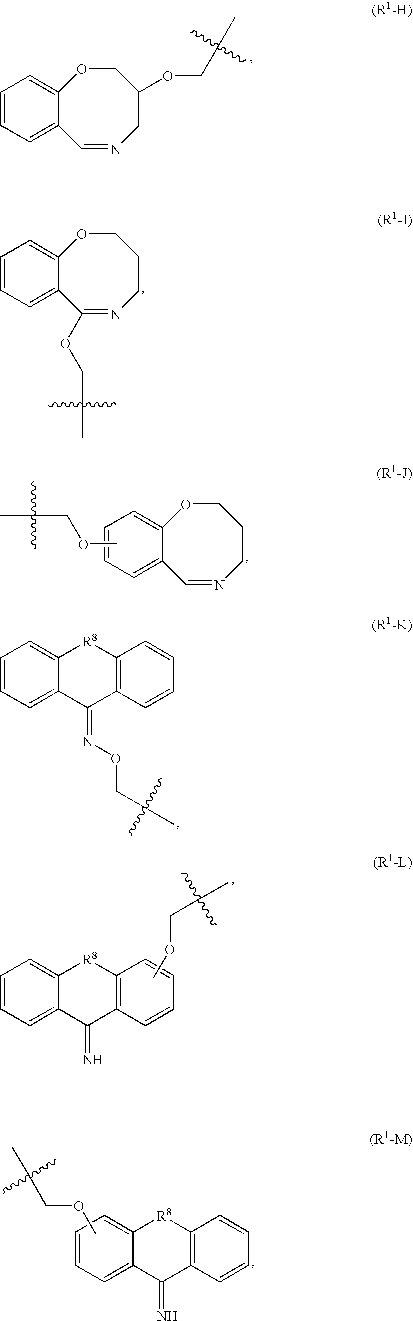 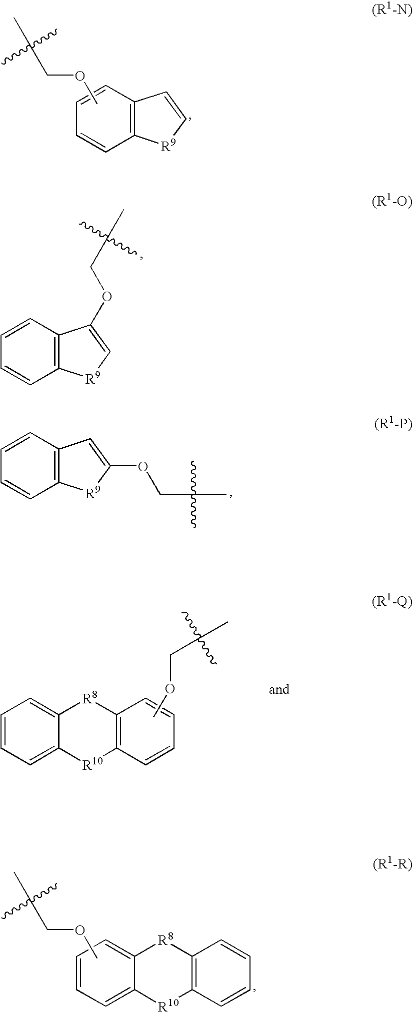
wherein
q is an integer selected from 1, 2 and 3,
R⁷ is O, C═O, S, NH or CH₂,
R⁸ is a bond, H₂C—CH₂, HC═CH, O, S or NH,
R⁹ is O, S or NH and
R¹⁰ is a bond or O.

46. The method of claim 44 wherein n is 1 and R⁵ is selected from the group consisting of: halo, cyano and a C₆-C₇ heterocycloalkyl having two points of attachment to ring A.

47. The method of claim 22 wherein R¹ is selected from the group consisting of:

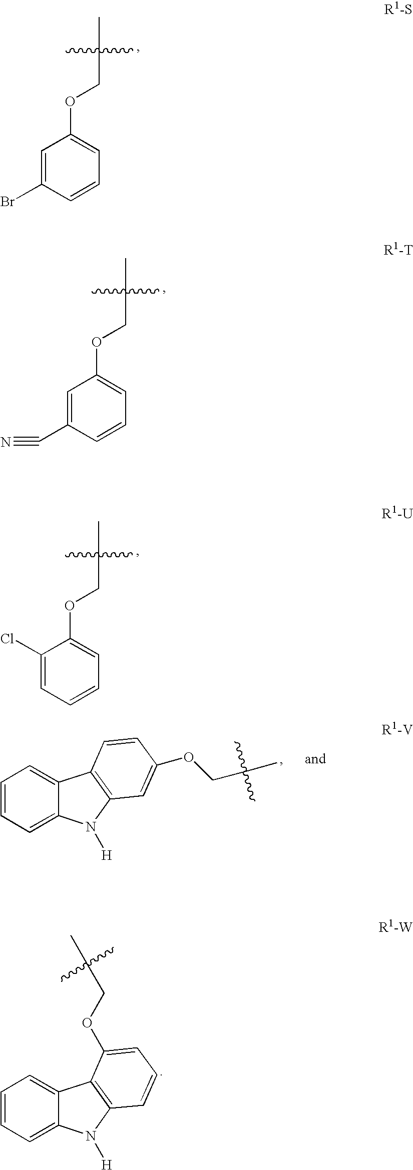

48. A method of making a compound comprising a beta-adrenergic inhibiting moiety and a linking moiety, the method comprising:
a) reacting 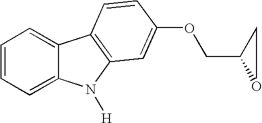
with an excess of NH₃, to form 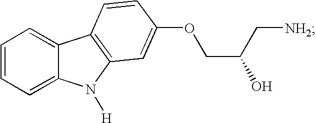
and
b) reacting 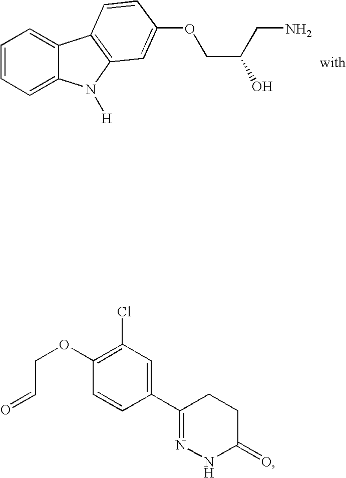
to form

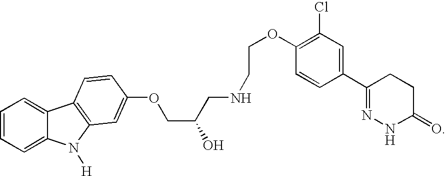

49. A method of making a compound comprising a beta-adrenergic inhibiting moiety and a linking moiety, the method comprising:
a) reacting 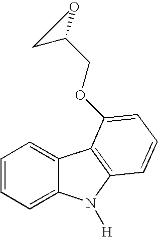
with an excess of NH₃, to form 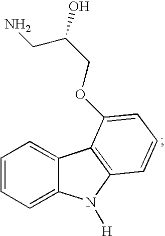
and
b) reacting 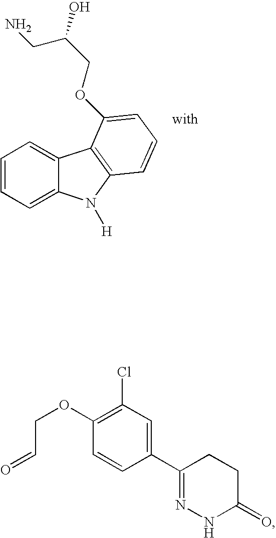
to form

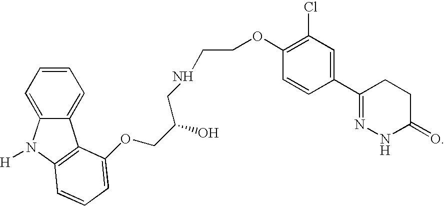

50. A method of making a compound comprising a beta-adrenergic inhibiting moiety and a linking moiety, the method comprising:
a) reacting 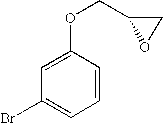
with an excess of NH₃, to form 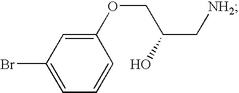
b) reacting 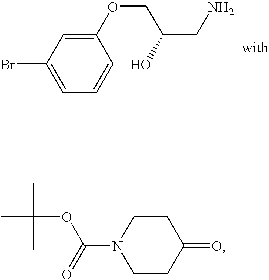
to form 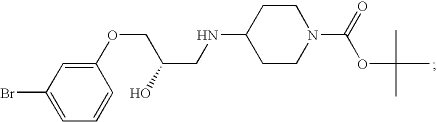
c) reacting 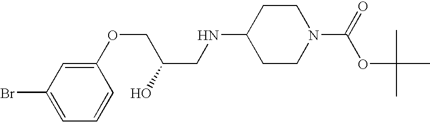
with HCl-THF to form 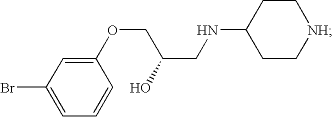
and
d) reacting 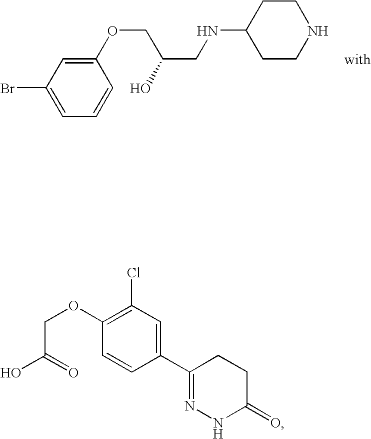
to form

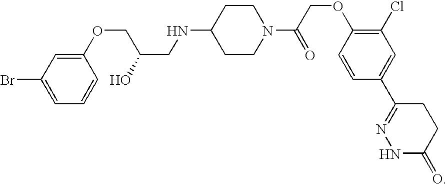

51. A method of making a compound comprising a beta-adrenergic inhibiting moiety and a linking moiety, the method comprising:
a) reacting 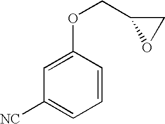
with an excess of NH₃, to form 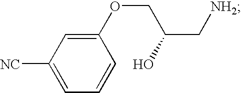
b) reacting 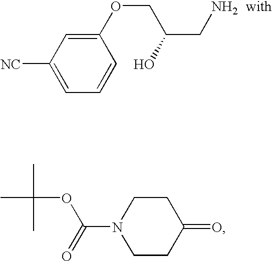
to form 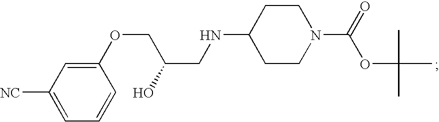
c) reacting 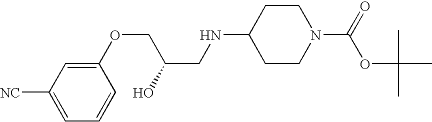
with HCl-THF to form 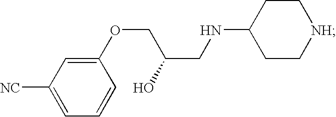
and
d) reacting 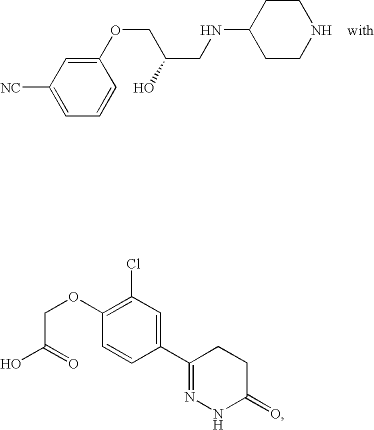
to form

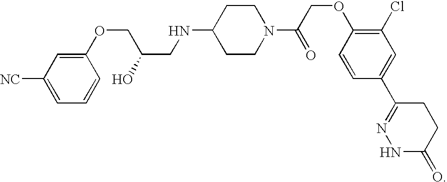

52. A method of making a compound comprising a beta-adrenergic inhibiting moiety and a linking moiety, the method comprising:
a) reacting 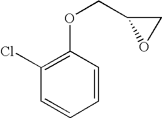
with an excess of NH₃, to form 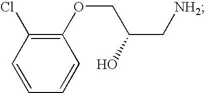
b) reacting 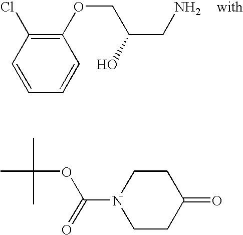
to form 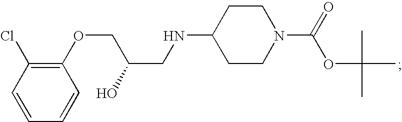
c) reacting 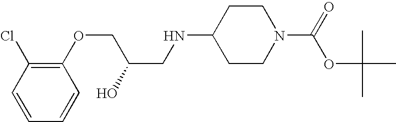
with HCl-THF to form 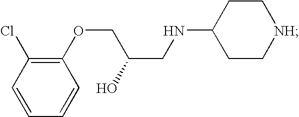
and
d) reacting 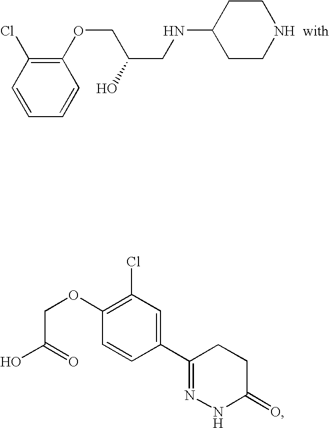
to form [chemical structure]
